(19) United States
(12) Patent Application Publication
(10) Pub. No.: US 2017/0199185 A1
(43) Pub. Date: Jul. 13, 2017

(54) Suspension of Gold-Coated Silver Nanoplates

(71) Applicant: Dai Nippon Toryo Co., Ltd., Osaka (JP)

(72) Inventors: Yuta Miyazawa, Tochigi (JP); Daigou Mizoguchi, Tochigi (JP)

(73) Assignee: Dai Nippon Toryo Co., Ltd., Osaka (JP)

(21) Appl. No.: 15/314,664

(22) PCT Filed: May 29, 2015
(86) PCT No.: PCT/JP2015/065658

§ 371 Date: Nov. 29, 2016

(30) Foreign Application Priority Data
May 30, 2014 (JP) 2014-112463
Nov. 14, 2014 (JP) 2014-231670

(51) Int. Cl. G01N 33/553; G01N 21/59; G01N 33/532; G01N 21/41; B22F 1/02; B22F 1/00

(52) U.S. Cl. CPC G01N 33/553; B22F 1/025; B22F 1/0022; B22F 1/0062; G01N 33/532; G01N 21/41; G01N 21/59; B22F 2301/255; B22F 2302/45; B22F 2303/01; G01N 2201/068; G01N 2021/5903

(57) ABSTRACT

A stable suspension of gold-coated silver nanoplates which is applicable to detection of various substances and able to accommodate various detection means and with which high detection sensitivity is attained in the detection of substances. This suspension of gold-coated silver nanoplates is characterized by containing 0-50 μM water-soluble polymer and having a pH of 10 or less.

### TECHNICAL FIELD

The present invention relates to a suspension of silver nanoplates coated with gold (hereinafter also referred to as gold-coated silver nanoplates).

### BACKGROUND ART

Since silver nanoplates absorb light through interaction with the light (localized surface plasmon resonance: LSPR), a suspension of silver nanoplates is known to exhibit a color according to the shape of the nanoplates. Further, it is also known that controlling the size and the shape of silver nanoplates can change light to be absorbed, that is, change the color.

Utilizing these properties, silver nanoplates have been used as labels on reagents for detecting test substances (for example, a label on an antibody used to detect a target protein).

On the other hand, silver nanoplates change their shapes when dissolved by oxidation. This change in shape of silver nanoplates due to oxidation may change an intended color.

Against this background, in order to stabilize silver nanoplates against oxidation, surfaces of silver nanoplates are coated with gold (Patent Literature 1 and Non Patent Literatures 1 and 2).

Meanwhile, diagnostic agents, which use a suspension of colored latex particles supporting a specific binding substance for a test substance, are known. Polystyrene particles having latex particle diameters of 0.02 to 2 μm and the like are known, which are colored in red or blue with an organic dye (Patent Literatures 2 and 3).

### CITATION LIST

### Patent Literatures

Patent Literature 1: Japanese Patent No. 3885054
Patent Literature 2: Japanese Patent No. 2677753
Patent Literature 3: Japanese Patent Publication No. 2006-266970

### Non Patent Literatures

Non Patent Literature 1: Materials Chemistry and Physics, 90 (2005), pp. 361-366
Non Patent Literature 2: Angew. Chern. Int. Ed., 2012, 51, pp. 5629-5633

### SUMMARY OF INVENTION

### Technical Problems

In the field of the detection technique in which gold-coated silver nanoplates are used as labels, there is a demand to enhance the sensitivity of detecting test substances. On the other hand, a suspension of silver nanoplates is susceptible to oxidation, which limits the applicable usage for detecting test Substances. Moreover, although a water-soluble polymer is sometimes added in order to stabilize a suspension of gold-coated silver nanoplates supporting a specific binding substance for a test substance, adding a large amount of such a water-soluble polymer decreases the sensitivity of the test-substance detection by the gold-coated silver nanoplates. Accordingly, an object of the present invention is to provide a stable suspension of gold-coated silver nanoplates, the suspension being applicable for detecting various test substances, being compatible with various detection means, and having a high sensitivity of detecting test substances.

### Solution to Problems

The present inventors have earnestly studied conditions for forming complexes of gold-coated silver nanoplates and detection reagents as well as the results of detecting test substances by using the obtained complexes. As a result, the inventors have found that a suspension of gold-coated silver nanoplates which has particular physical properties and composition achieves an enhancement in the sensitivity of detecting a test substance while the suspension retains the stability. Moreover, the present inventors have found that the gold-coated silver nanoplates are able to support specific binding substances for various test substances and are also compatible with various detection means. These findings have led to the completion of the present invention.

Specifically, the present invention provides a suspension, a method for detecting a test substance by using the suspension, and a kit including the suspension for use in the method, which are described below.

[1] A suspension of gold-coated silver nanoplates, the suspension comprising 0 to 50 μM of a water-soluble polymer and having a ph of 10 or less.
[2] The suspension according to [1], wherein an average thickness of gold on the gold-coated silver nanoplates is 1.0 nm or less.
[3] The suspension according to [1] or [2], wherein an average thickness of gold on the gold-coated silver nanoplates is 0.1 to 0.7 nm.
[4] The suspension according to any one of [1] to [3], wherein the concentration of the water-soluble polymer is 0 to 25 μM.
[5] The suspension according to any one of [1] to [4], wherein the pH is 4 to 10.
[6] The suspension according to any one of [1] to [5], wherein the pH is 5 to 9.
[7] The suspension according to any one of [1] to [6], wherein the gold-coated silver nanoplates support a specific binding substance for a test substance.
[8] The suspension according to [7], wherein a combination of the test substance and the specific binding substance, respectively, is selected from the group consisting of an antigen and an antibody capable of binding thereto, an antibody and an antigen capable of binding thereto, a sugar chain or a glycoconjugate and a lectin capable of binding to the sugar chain or the glycoconjugate, a lectin and a sugar chain or a glycoconjugate capable of binding to the lectin, a hormone or a cytokine and a receptor capable of binding to the hormone or the cytokine, a receptor and a hormone or a cytokine capable of binding to the receptor, a protein and a nucleic acid aptamer or a peptide aptamer capable of binding to the protein, an enzyme and a substrate capable of binding thereto, a substrate and an enzyme capable of binding thereto, biotin and avidin or streptavidin, avidin or streptavidin and biotin, IgG and Protein A or Protein G, Protein A or Protein G and IgG, and a first nucleic acid and a second nucleic acid capable of binding thereto.
[9] A method for detecting the test substance by using the suspension according to [7] or [8], the method comprising the steps of:

mixing the suspension with the test substance to form a complex of the test substance with the gold-coated silver nanoplates supporting the specific binding substance; and

detecting the complex.

[10] The method according to [9], wherein formation of the complex is detected by means selected from the group consisting of extinction measurement, absorbance measurement, turbidity measurement, particle size distribution measurement, particle diameter measurement, Raman scattering measurement, color-tone change observation, aggregate- or precipitate-formation observation, immunochromatography, electrophoresis, and flow cytometry.
[11] The method according to [9] or [10], wherein formation of the complex is detected by extinction measurement or absorbance measurement at an absorption wavelength of the gold-coated silver nanoplates within a range of 200 to 2500 nm.
[12] The method according to any one of [9] to [11], wherein formation of the complex is detected by extinction measurement or absorbance measurement at a maximum absorption wavelength of the gold-coated silver nanoplates within a range of 430 to 2000 nm.
[13] The method according to [9] or [10], wherein formation of the COmpleX is detected by tirbidity measurement in a wavelength region which is a long-wavelength side of a maximum absorption wavelength of the gold-coated silver nanoplates, and in which an extinction or absorbance is increased depending on the formation of the complex.
[14] A kit for use in the method according to any one of [9] to [13], the kit comprising the suspension according to [7] or [8].

### Advantageous Effects of Invention

The suspension of gold-coated silver nanoplates of the present invention is prepared as a stable suspension, and applicable for detecting various test substances with high sensitivity and also applicable to various detection means. Thus, the use of the suspension of the present invention enables accurate determinations in detecting a wide variety of test substances.

### BRIEF DESCRIPTION OF DRAWINGS

FIG. 1 shows chromaticity coordinates of yellow, magenta, and cyan, as well as chromaticity coordinates of suspensions N1, B1, and P1, in a CIE 1931 xy chromaticity diagram.

FIG. 2 shows chromaticity coordinates of red, blue, and green, as well as chromaticity coordinates of a mixed solution (B1+N1), a mixed solution (B1+P1), and a mixed solution (N1+P1), in a CIE 1931 xy chromaticity diagram.

FIG. 3 is a graph for illustrating optical properties of silver-nanoplate seed particles.

FIG. 4 is a figure showing a SEM observation image of the silver-nanoplate seed particles.

FIG. 5 is a graph for illustrating optical properties of aqueous suspensions a, b, c, and d of silver nanoplates.

FIG. 6 showing a SEM observation image of gold-coated silver nanoplates in a pH-adjusted, gold-coated silver nanoplate suspension B1.

FIG. 7 is a figure showing a SEM observation image of gold-coated silver nanoplates in a pH-adjusted, gold-coated silver nanoplate suspension N1.

FIG. 8 is a figure showing a SEM observation image of gold-coated silver nanoplates in a pH-adjusted, gold-coated silver nanoplate the suspension P1.

FIG. 9 is a figure showing a SEM observation image of gold-coated silver nanoplates in a pH-adjusted, gold-coated silver nanoplate the suspension R1.

FIG. 10 is a diagram for illustrating the procedure of an immunochromatographic test.

FIG. 11 is a graph showing the luminance analysis result of the immunochromatographic test.

FIG. 12 is a graph showing the luminance analysis result of the immunochromatographic test.

FIG. 13 is a graph showing the luminance analysis result of the immunochromatographic test.

FIG. 14 is a graph showing the luminance analysis result of the immunochromatographic test.

FIG. 15 is a graph showing the luminance analysis result of the immunochromatographic test.

FIG. 16 is a graph showing the luminance analysis result of the immunochromatographic test.

FIG. 17 is a graph showing the luminance analysis result of the immunochromatographic test.

FIG. 18 is a graph showing the luminance analysis result of the immunochromatographic test.

FIG. 19 is a graph showing the luminance analysis result of the immunochromatographic test.

FIG. 20 is a graph showing the luminance analysis result of the immunochromatographic test.

FIG. 21 is a graph showing the luminance analysis result of the immunochromatographic test.

FIG. 22 is a graph showing the luminance analysis result of the immunochromatographic test.

FIG. 23 is a graph showing the luminance analysis result of the immunochromatographic test.

FIG. 24 is a graph for illustrating a change in extinction when ConA was added to a suspension of gold-coated silver nanoplates supporting mannose.

FIG. 25 shows graphs for illustrating changes in extinction when HBs antigens were added to a suspension H2 (A), a suspension I2 (B), or a suspension J2 (C) of gold-coated silver nanoplates supporting an anti-HBs antigen antibody.

FIG. 26 is a graph for illustrating increases in extinction depending on turbidity.

FIG. 27 is a graph showing the luminance analysis result of an immunochromatography.

FIG. 28 is a graph showing the luminance analysis result of the immunochromatography.

### DESCRIPTION OF EMBODIMENTS

Hereinafter, the present invention will be described in more details.

A suspension of gold-coated silver nanoplates of the present invention is characterized by containing 0 to 50 μM of a water-soluble polymer and having a pH of 10 or less.

In the present invention, the term gold-coated silver nanoplate refers to a silver nanoplate (core) having a gold coating (shell) on the surface thereof.

Although silver nanoplates used as colored labels in the technical field of detecting test substances can be used in the present invention without particular limitations, preferable embodiments will be described in detail below.

The silver nanoplate refers to a nanoplate (plate) produced from silver.

The longest particle diameter (corresponding to the diameter in a case of a circular shape, and corresponding to the length of the longest side in a case of a triangular shape) of the main surface of the silver nanoplate is normally 10 to 1000 nm, preferably 10 to 150 nm. Further, the silver nanoplate has an aspect ratio (particle diameter/thickness) of normally 1.5 or more, preferably 1.5 to 10, so that the LSPR, absorption wavelength occurs in the visible light region, thereby enabling multicolor designs. For use in the detection with near-infrared light, plate-shaped silver nanoparticles having such an aspect ratio that LSPR occurs at 800 to 2000 nm should be used (for example, with an aspect ratio of 11, LSPR occurs around 900 nm).

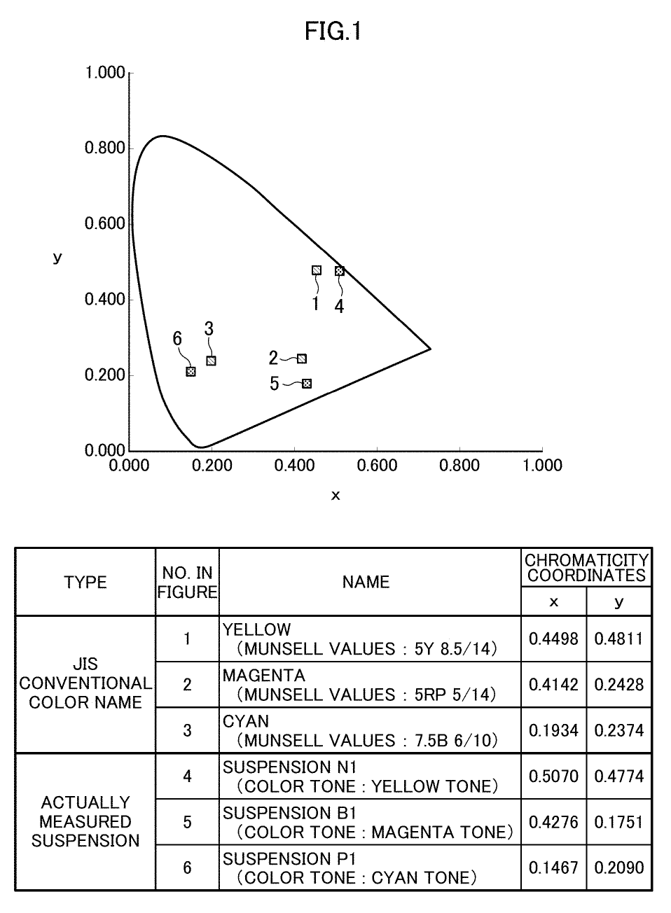

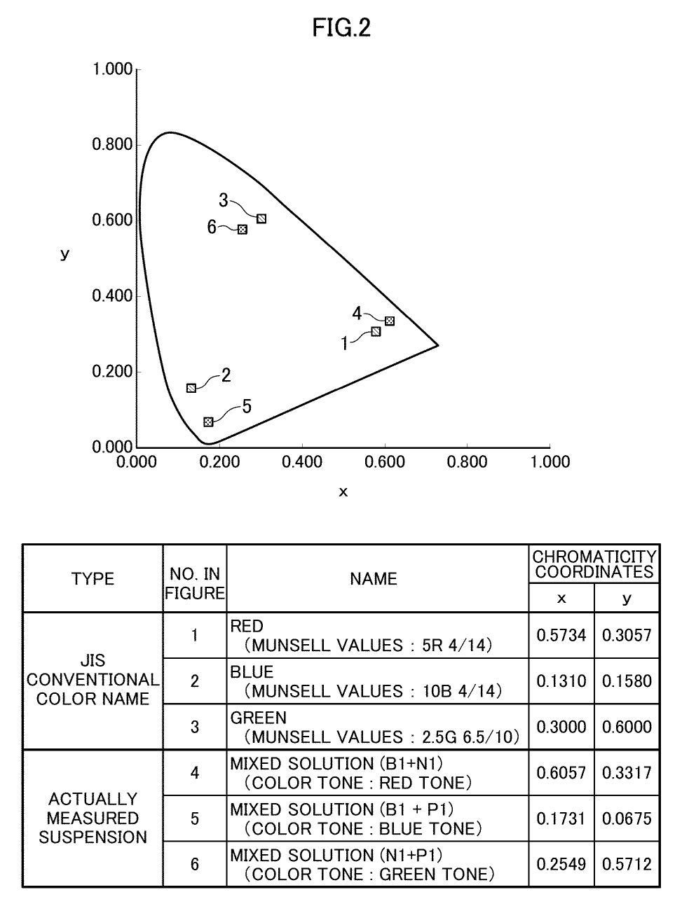

Adjusting the aspect ratios of plate-shaped silver nanoparticles makes it possible to select any LSPR absorption wavelength such as: yellow if the Munsell values of the Munsell color system, which is one of color space systems quantitatively representing colors (hereinafter also referred to simply as Munsell values), are 5Y 8.5/14 and the coordinates in a CIE 1931 xy chromaticity diagram (hereinafter also referred to simply as chromaticity coordinates) are x: 0.4498 and y: 0.4811 (yellow-based color of plate-shaped silver nanoparticles with LSPR around 400 to 500 nm); magenta if the Munsell values are 5RP 5/14 and the chromaticity coordinates are x: 0.4142 and y: 0.2428 (magenta-based color of plate-shaped silver nanoparticles with LSPR occurring around 500 to 600 nm); and cyan if the Munsell values are 7.5 B 6/10 and the chromaticity coordinates are x: 0.1934 and y: 0.2374 (cyan-based color of plate-shaped silver nanoparticles with LSPR occurring around 600 to 750 nm). Note that FIG. 1 shows the chromaticity coordinates of yellow, magenta, and cyan in a CIE 1931 xy chromaticity diagram. Colors may be designed by mixing two or more types of plate-shaped silver nanoparticles having different aspect ratios from each other. For example, it is possible to design: red (Munsell values: 5R 4/14, chromaticity coordinates of x: 0.5734 and y: 0.3057) by mixing yellow with magenta; blue (Munsell values: 10B 4/14, chromaticity coordinates of x: 0.1310 and y: 0.1580) by mixing magenta with cyan; green (Munsell values: 2.5G 6.5/10, chromaticity coordinates of x: 0.3000 and v: 0.6000) by mixing yellow with cyan; and other colors. Note that FIG. 2 shows the chromaticity coordinates of red, blue, and green in a CIE 1931 xy chromaticity diagram. Further, multicolor designs can be employed by applying subtractive mixing using, as three primary colors, three or more types of plate-shaped silver nanoparticles having different aspect ratios from each other and exhibiting a yellow-based color, a magenta-based color, and a cyan-based color. For example, when black is designed by mixing plate-shaped silver nanoparticles of a yellow-based color, a magenta-based color, and a cyan-based color at certain ratios, the contrast difference in the event of an immunochromatographic test is so large between a test substance detection line and the background (white) that the visibility (detection sensitivity) is enhanced.

The thicknesses of the silver nanoplates are not particularly limited, as long as plasmon absorption is possible. The thicknesses are normally 40 nm or less, preferably 5 to 20 nm.

The shapes of the silver nanoplates are not particularly limited, as long as plasmon absorption is possible. The shape can be selected depending on an intended color. Specific examples of the shapes include polygonal shapes such as triangular shapes, pentagonal shapes, and hexagonal shapes; circular shapes having curved corners; and the like.

In the present invention, a single type of silver nanoplates (uniform shape) may be used, or multiple types of silver nanoplates having different shapes from each other may be used in mixture.

The shapes and the sizes (maximum lengths of the main surfaces) of the silver nanoplates can be set as appropriate depending on an intended color or maximum absorption wavelength. The maximum absorption wavelength of the silver nanoplate may be adjusted within a range of 430 to 2000 nm, preferably 430 to 1500 nm, and may be adjusted within a range of particularly preferably 430 to 1000 nm. A relation of a color to the sizes and the shapes of silver nanoplates is described in, for example, Published Japanese Translation of PCT International Application No. 2011-508072. For example, if silver nanoplates have triangular and hexagonal shapes (the maximum lengths of the main surfaces: 20 nm, thicknesses: 5.1 nm), magenta (maximum absorption wavelength: 538 nm) can be exhibited. The maximum absorption wavelength of silver nanoplates is stabilized after coating of formed silver nanoplates with gold, pH adjustment of the suspension, and/or supporting of a specific binding substance for a test substance.

As the silver nanoplates, commercially available products may be used, or silver nanoplates produced according to known production methods or the method described later in Examples may be used.

The thickness of gold coating the surfaces of the silver nanoplates is not particularly limited, as long as the color developing ability of the silver nanoplates is not influenced. Nevertheless, the average thickness is preferably 1.0 nm or less, more preferably 0.7 nm or less. The gold coating having a thickness of 1.0 nm or less makes it possible to suppress oxidation of the silver nanoplates while the silver nanoplates retain the plasmon absorption.

The lower limit of the thickness of the gold is not particularly limited, as long as the object of coating the silver nanoplate surfaces with gold can be achieved. Nevertheless, the average thickness is preferably no less than 0.1 nm, or 0.3 nm. Note that, in the present invention, the average thickness of the gold may be calculated from thicknesses of gold on the silver nanoplate surfaces measured by adopting high-angle annular dark field scanning transmission electron microscopy (HAADF-STEM). To be more specific, the calculation includes: selecting any ten particles from a HAADF-STEM image; measuring any ten sites on each of the particles to obtain data on thicknesses of gold at 100 sites in total; and excluding the highest and lowest 10% of the data to thus take the average of 80 sites as an average thickness of gold.

The coating method is not particularly limited, as long as the object of coating the silver nanoplate surfaces with gold can be achieved. Thus, it is possible to adopt known coating methods or the coating method described later in Examples.

The gold-coated silver nanoplates of the present invention may have gold entirely coating the surfaces of the silver nanoplates, or may have cold partially coating the surfaces of the silver nanoplates. Whether the surfaces of the silver nanoplates are entirely or partially coated with gold can be checked by various methods normally used such as observations with an electron microscope and measurements of physicochemical properties. For example, when the surfaces of the silver nanoplates are entirely or partially coated with gold, the stability against acids or sodium or chloride ions is increased, so that the resultant is stable against oxidation. As a result, even in a case where the spectral properties (maximum absorption wavelength) of a suspension of the silver nanoplates after the gold-coating treatment are measured under harsh conditions for silver nanoplates, for example, in an acidic solution (for example, 2% hydrogen peroxide) or a buffer solution (for example, 10 mM phosphate buffer saline (with or without divalent ions)), if the spectral properties change only slightly in comparison with the measurement in water, it can be determined that the silver nanoplates are coated with gold.

In addition, whether the silver nanoplates are coated with gold can also be checked by measuring the concentrations of gold and silver in a suspension of the gold-coated silver nanoplates. To be more specific, as illustrated in the following procedure, after a suspension is centrifuged, the supernatant is removed and the resulting precipitate is suspended again in ultrapure water in the same amount as that of the removed supernatant. Then, aqua regia is added to the suspension, followed by boiling. The resulting solution was analyzed by using an ICP emission spectrometer.

1. A suspension of gold-coated silver nanoplates is centrifuged (25,000 rpm, 26,000 g). Then, the supernatant is removed, and the resulting precipitate is suspended again in ultrapure water in the same amount as that of the removed supernatant.

2. Aqua regia is added to the suspension obtained in step 1 above, followed by boiling for 5 minutes. Thereby, gold and silver are dissolved into aqua regia.

3. The solution obtained in step 2 above is measured using an ICP emission spectrometer.

Note that the concentration of each metal is calculated from a calibration curve which is created by measuring a standard sample of a certain concentration in the same manner as described above.

The obtained concentration of each metal reveals the ratio of gold and silver. Moreover, coating of silver nanoplate surfaces with gold and the thickness of the gold can be checked. For example, the thickness of gold on triangular silver nanoplates can be calculated as follows.

1. ICP emission spectroscopy result (example)

Gold concentration:silver concentration=1:4

2. Volume of silver nanoplate (with a shape: equilateral triangle, height: 30 nm, thickness: 8 nm)

$$\begin{matrix}( area of triangle ) \times ( thickness ) = ( 30 nm \times ( 30 \times 2 \div √ 3 ) nm \div 2 ) \times 8 nm \\ = 4157 {nm}^{3} ( = 4157 \times {10}^{- 21} {cm}^{3} )\end{matrix}$$

3. Relative density of silver

10.51 g/cm³

4. Mass of triangular silver nanoplate

$$( volume of triangular silver nanoplate ) \times ( relative density of silver ) = ( 4157 \times {10}^{- 21} {cm}^{3} ) \times 10.51 g / {cm}^{3} = 4.37 \times {10}^{- 17} g$$

5. Mass (X) of gold coating triangular silver nanoplate

1:4=X:4.37×10⁻¹⁷ g

X=1.09×10⁻¹⁷ g

6. Relative density of gold

19.32 g/cm³

7. Volume of gold coating triangular silver nanoplate

$$( mass of gold ) + ( relative density of gold ) = 1.09 \times {10}^{- 17} g \div 19.32 g / {cm}^{3} = 5.64 \times {10}^{- 19} {cm}^{3} ( = 564 {nm}^{3} )$$

8. Surface area of triangular silver nanoplate

$$( areas of triangle ) + ( areas of side surfaces of particle ) = \begin{matrix}( 30 \times ( 60 \div √ 3 ) \\ \end{matrix} \div 2 ) \times 2 + ( 8 \times ( 60 \div √ 3 ) ) \times 3 = 1871 {nm}^{2}$$

9. Thickness of gold coating triangular silver nanoplate

$$( volume of gold coating triangular silver nanoplate ) \div ( surface area of triangular silver nanoplate ) = 564 {nm}^{3} \div 1871 {nm}^{2} = 0.30 nm ( = 3.0 Å )$$

As described above, if the result of the analysis using an ICP emission spectrometer revealed that the ratio of the gold concentration and the silver concentration is 1:4, it can be calculated that the thickness of gold on the surfaces of the silver nanoplates (in the case of the particle having a shape: equilateral triangle, height: 30 nm, thickness 8 nm) having been subjected to the gold-coating treatment is 0.30 nm. This means that the silver nanoplates are coated with one or two layers of gold atoms.

Note that, in the present invention, the term “gold coating the surfaces of the silver nanoplates” refers to gold present on the surfaces of the silver nanoplate, but also includes gold present in the form of alloy with silver, in addition to gold present alone.

As the gold-coated silver nanoplates, commercially available products may be used, or gold-coated silver nanoplates produced according to known production methods or the method described later in Examples may be used.

In the present invention, a single type of gold-coated silver nanoplates may be used, or multiple types of gold-coated silver nanoplates having different shapes and sizes from each other may be used in mixture.

In the suspension of gold-coated silver nanoplates of the present invention (hereinafter also referred to as the suspension of the present invention), solid gold-coated silver nanoplates are suspended in a liquid dispersion medium.

Any dispersion medium can be used without particular limitations, as long as it is capable of dispersing the gold-coated silver nanoplates. Specific examples thereof include water, aqueous buffer solutions (such as a phosphate buffer saline, a Tris-HCl buffer solution, a HEPES buffer solution), alcohols such as ethanol and methanol, ketones such as acetone and methyl ethyl ketone, tetrahydrofuran, and the like. The dispersion medium is preferably water because it is suitable in biochemical experiments.

The dispersion medium may be of a single type or may be a mixture of multiple types.

In the suspension of gold-coated silver nanoplates, the gold-coated silver nanoplates are dispersed in the dispersion medium while left standing. The gold-coated silver nanoplates are precipitated when left standing, but may be dispersed in the dispersion medium by shaking or ultrasonic dispersion treatment.

As the suspension of gold-coated silver nanoplates, a suspension obtained as a result of carrying out a method for producing gold-coated silver nanoplates may be used (provided that the requirements of water-soluble polymer concentration and ph to be described later are satisfied), or the gold-coated silver nanoplates may be isolated from this suspension and then dispersed in a dispersion medium.

The method for producing the suspension is not particularly limited. It is possible to adopt known production methods or the production method described later in Examples.

The suspension of the present invention has a silver content of preferably 50% by mass or less, more preferably 1 to 0.000015% by mass, and particularly preferably 0.1 to 0.000015% by mass, relative to the total mass of the suspension. The suspension having a silver content of 50% by mass or less has effects that the dispersion stability is improved; and when used in a biochemical test (for example, an immunochromatographic test) utilizing the spectral properties, a color development is easily checked (for example, a detection line is visually checked in the immunochromatographic test).

The suspension of the present invention may optionally contain or may not contain a water-soluble polymer, and the concentration is within a range of 0 to 50 μM.

In the present invention, the term water-soluble polymer refers to a water-soluble substance having a molecular weight of 500 to 1,000,000, preferably 500 to 100,000. In the present invention, the term water-soluble means that 0.001% by mass or more of the polymer is dissolved in water at normal temperature and normal pressure.

Specific examples of the water-soluble polymer include polyvinylpyrrolidone (PVP), polyethylene glycols, polyacrylamides, polyvinyl alcohols, polyacrylic acids, polymethacrylic acids, polyallylamines, dextrans, polymethacrylamides, polyvinylphenols, polyvinylbenzoates, bovine serum albumins (BSA), caseins, bis(p-sulfonatophenyl)phenylphosphine, polystyrenesulfonic acids, and the like. Commercially available dispersants which are used in paints, inks, and so forth and contain these water-soluble polymers may be used as the water-soluble polymer of the present invention. Moreover, the aforementioned specific examples of the water-soluble polymer may be modified with a functional group such as a hydroxy group, a mercapto group, a disulfide group, an amino group, or a carboxyl group, which chemically bond to the fine metal particles in the suspension of the present invention.

The type of the water-soluble polymer contained in the suspension of the present invention can be selected depending on the usage of the suspension. For example, when the suspension is used in an in vivo experiment or a cell experiment, polyvinylpyrrolidone, polyethylene glycols, polyvinyl alcohols, and the like, which have favorable biocompatibilities, may be used.

In the suspension of the present invention containing the water-soluble polymer, the concentration of the water-soluble polymer dissolved or dispersed in the suspension (i.e., the number of moles of the water-soluble polymer per liter of the suspension) is 50 μM or less, preferably 25 μM or less, and particularly preferably 10 μM or less. The water-soluble polymer at a concentration of 50 μM or less makes it possible to enhance the test-substance detection sensitivity of the suspension of gold-coated silver nanoplates supporting a specific binding substance for a test substance of the present invention, in comparison with a case where the concentration exceeds 50 μM.

Moreover, in another preferable embodiment, the suspension of the present invention does not contain the water-soluble polymer (i.e., 0 μM). Without the water-soluble polymer, the cold-coated silver nanoplates of the present invention are capable of forming a stable suspension by supporting a specific binding substance for a test substance. Thus, the suspension of the present invention does not particularly have to contain the water-soluble polymer in order to enhance the sensitivity of detecting a test substance.

Although the present invention is not bound by any particular theory, it is believed that when the concentration of the water-soluble polymer is 50 μM or less, for example, a specific binding substance for a test substance to be described later is favorably supported by the gold-coated silver nanoplates, or the gold-coated silver nanoplates supporting a specific binding substance for a test substance form a stable complex with the test substance; as a result, the sensitivity of detecting the test substance is enhanced.

The method for measuring the water-soluble polymer concentration includes nuclear magnetic resonance spectroscopy (NMR), size-exclusion chromatography (SEC), gel-filtration chromatography (GPC), thermogravimetric-differential thermal analysis (TG-DTA), and the like.

In NMR, the suspension of gold-coated silver nanoplates is dried, and the resulting solid content is dissolved (dispersed) in a deuterated solvent for the measurement. In the deuterated solvent, an internal standard substance (for example, maleic acid) of a known concentration has been added in advance, and the water-soluble polymer concentration can be calculated by comparing integrated values of signals derived from the water-soluble polymer with integrated values of signals derived from the internal standard substance in the resulting spectrum.

Moreover, in GPC, first, multiple aqueous solutions of the water-soluble polymer of known concentrations are analyzed, and a calibration curve is created from peak areas derived from the water-soluble polymer in the obtained chart. Next, the suspension of gold-coated silver nanoplates is measured. The water-soluble polymer concentration can be calculated from the peak areas derived from the water-soluble polymer in the obtained chart.

Further, in TG-DTA, the amount of the water-soluble polymer can be calculated from a change in weight when the suspension of gold-coated silver nanoplates is measured for the dried solid content.

The suspension of the present invention has a pH of 10 or less, preferably 4 to 10 or 5 to 10, and particularly preferably 5 to 9 or 6 to 9. The pH of 10 or less enhances the test-substance detection sensitivity of the suspension of gold-coated silver nanoplates supporting a specific binding substance for a test substance to be described later.

Although the present invention is not bound by any particular theory, it is believed that when the suspension has a pH of 10 or less, for example, a specific binding substance for a test substance to be described later is favorably supported by the gold-coated silver nanoplates, or the gold-coated silver nanoplates supporting a specific binding substance for a test substance form a stable complex with the test substance; as a result, the sensitivity of detecting the test substance is enhanced.

In the present invention, the pH of the suspension refers to a pH at room temperature (20 to 30° C.) measured by general pH measurement methods adopted in this technical field, for example, a glass electrode method.

To adjust the pH of the suspension, a pH adjuster may be added to the suspension. As the pH adjuster, known substances can be used. Specific examples thereof include PBS buffer solutions, sodium carbonate, sodium hydroxide, citric acid, and the like.

The suspension of the present invention can be used to label a specific binding substance for a test substance, that is, a detection reagent. In other words, the gold-coated silver nanoplates of the present invention can support a specific binding substance for a test substance. The term “support” means that the gold-coated silver nanoplates are linked to a specific binding substance for a test substance to form a complex, regardless of the linking mode such as a covalent bond, a non-covalent bond, or direct or indirect linking. As the supporting method, normal supporting methods can be adopted without particular limitations. It is possible to adopt: a method in which the gold-coated silver nanoplates are directly linked to a specific binding substance for a test substance by utilizing physical adsorption, chemical adsorption (covalent bond to the surface), chemical bond (covalent bond, coordinate bond, ionic bond, or metallic bond), or the like; and a method in which a portion of the above-described water-soluble polymer is linked to the surfaces of the gold-coated silver nanoplates, and then a specific binding substance for a test substance is directly or indirectly linked to an end, or a main chain or a side chain, of the water-soluble polymer. For example, in a case where the specific binding substance for the test substance is an antibody, the suspension of the present invention is mixed with a solution of the antibody, shaken, and centrifuged, so that the gold-coated silver nanoplates supporting the antibody (labeled detection reagent) can be obtained as a precipitate.

As the specific binding substance for the test substance of the present invention, any specific binding substance can be used without particular limitations, as long as it is capable of detecting a detection target of a test substance through complex formation with the test substance and capable of utilizing the gold-coated silver nanoplates as a label. Specific examples of a combination of the test substance and the specific binding substance for the test substance include an antigen and an antibody capable of binding thereto, a sugar chain or a glycoconjugate and a lectin capable of binding to the sugar chain or the glycoconjugate, a lectin and a sugar chain or a glycoconjugate capable of binding to the lectin, a hormone or a cytokine and a receptor capable of binding to the hormone or the cytokine, a receptor and a hormone or a cytokine capable of binding to the receptor, a protein and a nucleic acid aptamer or a peptide aptamer capable of binding to the protein, an enzyme and a substrate capable of binding thereto, a substrate and an enzyme capable of binding thereto, biotin and avidin or streptavidin, avidin or streptavidin and biotin, IgG and Protein A or Protein G, Protein A or Protein G and IgG, a first nucleic acid and a second nucleic acid capable of binding (hybridizing) thereto, and the like. The second nucleic acid may be a nucleic acid containing a sequence complementary to that of the first nucleic acid.

In the case where the test substance is an antigen, the specific binding substance for the antigen may be an antibody. The antibody may be a polyclonal antibody, a monoclonal antibody, a single chain antibody, or fragments thereof, all of which are capable of specifically binding to the antigen. The fragments may be a F(ab) fragment, a F(ab′) fragment, a F(ab′)₂ fragment, or a F(v) fragment. The antigen serving as a test substance may be a lectin such as concanavalin A (ConA), wheat germ agglutinin, and ricin; may be a virus such as influenza viruses, adenoviruses, RS virus, rotaviruses, human papillomaviruses, human immunodeficiency viruses, and hepatitis B virus, or substances thereof (for example, hepatitis B virus antigens (HBs antigens) or influenza virus hemagglutinins); may be a pathogenic microorganism such as Aspergillus flavus, Chlamydia, Treponema pallidum, Streptococcus hemolyticus, Bacillus anthracis, Staphylococcus aureus, Shigella, Escherichia coli, Salmonella, Salmonella typhimurium, Salmonella paratyphi A, Pseudomonas aeruginosa, and Vibrio parahaemolyticus, or substances thereof (for example, Aspergillus flavus aflatoxin (B1, B2, G1, G2, M1, or the like) enterohemorrhagic Escherichia coli verotoxin, or Streptococcus hemolyticus streptolysin O); may be a blood protein such as immunoglobulin G (IgG), rheumatoid factor, and C-reactive protein (CRP); may be a glycoprotein such as mucins; may be a hormone such as insulin or a pituitary hormone (for example, growth hormone (GH), adrenocorticotropic hormone (ACTH), thyroid-stimulating hormone (TSH), follicle-stimulating hormone (FSH), luteinizing hormone (LH), prolactin, or melanocyte-stimulating hormone (MSH)), a thyrotropin-releasing hormone (TRH), a thyroid hormone (for example, diiodothyronine or triiodothyronine), a chorionic gonadotropin, a calcium metabolism regulating hormone (for example, calcitonin or parathormone), a pancreatic hormone, a gastrointestinal hormone, a vasoactive intestinal peptide, a follicle hormone (for example, estrone), a natural or synthetic corpus luteum hormone (for example, progesterone), a male sex hormone (for example, testosterone), and an adrenal cortical hormone (for example, cortisol); may be other in vivo substances such as serotonin, urokinase, ferritin, substance P, prostaglandins, and cholesterols; may be a tumor marker such as prostatic acid phosphatase (PAP), prostate-specific antigen (PSA), alkaline phosphatase, transaminase, trypsin, pepsinogen, α-fetoprotein (AFP), and carcinoembryonic antigen (CEA); may be a sugar chain antigen such as a blood group antigen;

or may be a marker, such as hemoglobin and transferrin, used in the detection of fecal occult blood. Moreover, in the case where the antigen serving as a test substance is an in vivo substance such as a hormone or a cytokine, the specific binding substance for the in vivo substance may be not only an antibody but also a receptor. In the case where the antigen serving as a test substance is a sugar chain or a glycoconjugate having a sugar chain, the specific binding substance for the sugar chain or the glycoconjugate having a sugar chain may be not only an antibody but also a lectin. Additionally, the antigen serving as a test substance may be a hapten such as penicillin and cadmium.

In the case where the test substance is an antibody, the specific binding substance for the antibody may be an antigen. The antigen may be a complete antigen or a fragment thereof which are capable of specifically binding to the antibody, or may be a fusion substance in which a complete antigen and a fragment thereof binds to another carrier. The antibody serving as a test substance may be an autoantibody such as an anti-cyclic citrullinated peptide (CCP) antibody or an anti-phospholipid antibody, or may be an antibody against an exogenous antigen, such as an anti-Chlamydia antibody, an anti-HIV antibody, or an anti-HCV antibody.

In the case where the test substance is a sugar chain, the specific binding substance for the sugar chain may be a lectin. The lectin may be a galectin, a C-type lectin, a legume lectin, or fragments thereof, all of which are capable of specifically binding to the sugar chain. The sugar chain serving as a test substance may be a monosaccharide or a polysaccharide, or may be a glycoconjugate in which a monosaccharide or a polysaccharide is linked to a protein or a lipid. For example, in a case where the test substance is a sugar chain containing mannose, a legume lectin concanavalin A (ConA) can be used as the specific binding substance for the sugar chain.

In the case where the test substance is a lectin, the specific binding substance for the lectin may be a sugar chain. The sugar chain may be a monosaccharide, a polysaccharide, or a glycoconjugate, all of which are capable of specifically binding to the lectin, or a fusion substance in which a monosaccharide, a polysaccharide, or a glycoconjugate binds to another carrier. The lectin serving as a test substance may be a galectin, a C-type lectin, or a legume lectin. For example, in a case where the test substance is a legume lectin concanavalin A (ConA), a sugar chain containing mannose can be used as the specific binding substance for the lectin.

In the case where the combination of the test substance and the specific binding substance for the test substance is a protein and a nucleic acid aptamer or a peptide aptamer capable of binding to the protein, the nucleic acid aptamer may be, for example, a DNA aptamer capable of binding to: a bacterium such as Bacillus anthracis, Staphylococcus aureus, Shigella sonnei, Escherichia coli, Salmonella typhimurium, Streptococcus hemolyticus, Salmonella paratyphi A, Staphylococcal enterotoxin B, Pseudomonas aeruginosa, or Vibrio parahaemolyticus; a tumor darker expressed on the epithelial cell surface, such as mucin 1; or an enzyme such as β-galactosidase or thrombin; or the like. Alternatively, the nucleic acid aptamer may be an RNA aptamer capable of binding to a Tat protein or a Rev protein of human immunodeficiency virus. The peptide aptamer may be, for example, a peptide aptamer capable of binding to an oncoprotein HPV16 E6 of human Papillomavirus (HPV).

In a case where the test substance is a vitamin, the specific binding substance for the vitamin may be a vitamin binding protein such as transcalciferin capable of binding to vitamin D and a transcobalamin capable of binding to vitamin B12. In a case where the test substance is an antibiotic, the specific binding substance for the antibiotic may be a penicillin-binding protein such as PBP1 and PBP2 capable of binding to penicillin.

The nucleic acid serving as a test substance or a substance capable of binding to the test substance may be, for example, a DNA, an RNA, an oligonucleotide, a polynucleotide, or amplification products thereof.

The suspension of the present invention may contain an optional component, as long as the gold-coated silver nanoplates are not adversely influenced. Such an optional component includes reagents (for example, sodium borohydride and ascorbic acid) used in carrying out the method for producing gold-coated silver nanoplates, dispersants (for example, trisodium citrate), and the like.

The gold-coated silver nanoplates supporting the specific binding substance for the test substance of the present invention can be used to detect the corresponding test substance.

The method for detecting the test substance of the present invention includes the steps of: mixing the suspension of the present invention with the test substance to form a complex of the test substance with the gold-coated silver nanoplates supporting the specific binding substance for the test substance; and detecting the complex.

The complex can be detected without particular limitations by utilizing means normally used in the field of detecting test substances or means normally used to detect aggregates or precipitates. For example, formation of the complex may be detected by means selected from the group consisting of extinction measurement, absorbance measurement, turbidity measurement, particle size distribution measurement, particle diameter measurement, Raman scattering measurement, color-tone change observation, aggregate- or precipitate formation observation, immunochromatography, electrophoresis, and flow cytometry.

In immunochromatography, gold-coated silver nanoplates gather at a detection site (in the form of line). In this event, when the gold-coated silver nanoplates absorb light in the visible region, the formation of the complex can be recognized as a color. The result of this test can be quantified by visual judgment and luminance difference analysis. In the visual judgment, whether or not the detection line is present after the immunochromatographic test is visually checked, and the lowest concentration of a test substance at which the detection line can be checked may be regarded as the detection sensitivity. In the luminance analysis, when an absolute value of a value obtained by subtracting a luminance difference 2 from a luminance difference 1, which are as described below, is 2 (detection limit luminance difference) or larger, the lowest concentration of a test substance may be regarded as the detection sensitivity.

1. The luminance difference between a detection line and a section other than the detection line when an immunochromatographic test is conducted using a developing solution containing no test substance.

2. The luminance difference between a detection line and a section other than the detection line on an immunochromatographic support when an immunochromatographic test is conducted using a developing solution containing a test substance.

Meanwhile, in an immunochromatographic test using gold-coated silver nanoplates which absorb light in the near-infrared region, the detection portion is irradiated with near-infrared light after the test. The light absorption in this event is measured, so that the detection can be checked.

Next, the effects of the present invention will be described specifically by way of Examples. However, the present invention is not limited to Examples.

### EXAMPLES

### Example 1

### (1) Preparation of Silver-Nanoplate Seed Particles

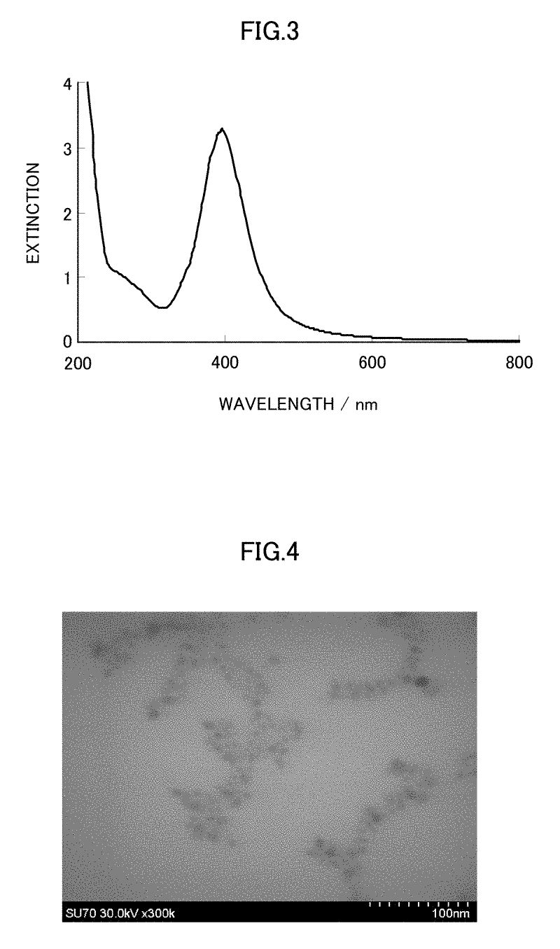

To 20 mL of an aqueous solution of 2.5 mM sodium citrate, 1 mL of an aqueous solution of 0.5 g/L polystyrenesulfonic acid having a molecular weight of 70,000 and 1.2 mL of an aqueous solution of 10 mM sodium borohydride were added. Then, 50 mL of an aqueous solution of 0.5 mM silver nitrate was added thereto with stirring at 20 mL/min. The obtained solution was left standing in an incubator (30° C.) for 60 minutes. Thereby, an aqueous suspension of silver-nanoplate seed particles was prepared. FIG. 3 shows optical properties of the prepared aqueous suspension (raw solution). The optical properties were measured using a UV-Vis-NIR spectrometer MPC3100UV-3100PC manufactured by Shimadzu Corporation under conditions of an optical path length: 1 cm and a measurement wavelength: 190 to 1300 nm. The maximum absorption was exhibited at a wavelength of 396 nm (extinction 3.3), at which LSPR of spherical silver nanoparticles occurs. Note that the extinction of the present invention refers to a value of absorbance when a suspension is measured with a spectrometer. Additionally, FIG. 4 shows a SEM observation image. For the analysis of the SEM observation image, a scanning electron microscope SU-70 manufactured by Hitachi, Ltd. was used. The plate-shaped particles had particle diameters of mainly 3 nm or more but less than 10 nm.

### (2) Preparation of Silver Nanoplates (Magenta)

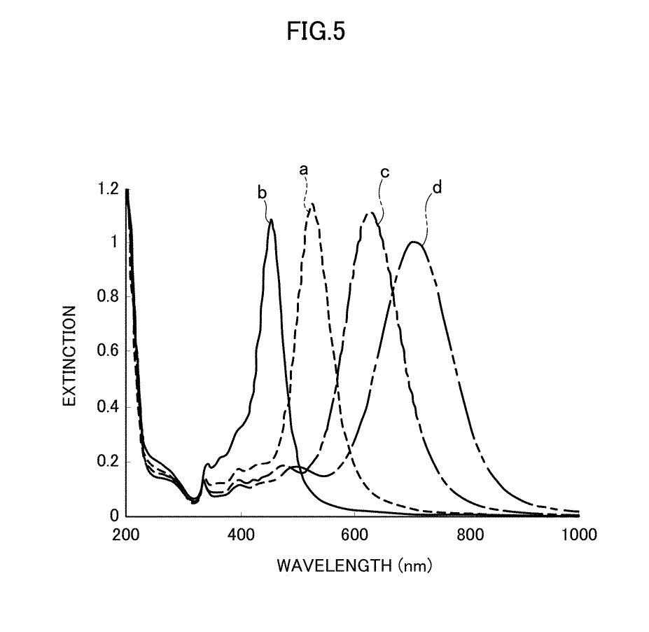

To 200 mL of ultrapure water, 4.5 mL of an aqueous solution of 10 mM ascorbic acid was added, and further 4 mL of the aqueous suspension of silver-nanoplate seed particles prepared in (1) was added. To the obtained solution, 120 mL of an aqueous solution of 0.5 mM silver nitrate was added with stirring at 30 mL/min. The stirring was stopped four minutes after the addition of the aqueous solution of silver nitrate was completed. Then, 20 mL of an aqueous solution of 25 mM sodium citrate was added thereto. The obtained solution was left standing in an incubator (30° C.) in an air atmosphere for 100 hours. Thereby, an aqueous suspension a of silver nanoplates was prepared. The prepared suspension was 4-fold diluted by volume with ultrapure water. FIG. 5 shows optical properties of the aqueous suspension. The maximum absorption was exhibited at a wavelength of 526 nm (extinction 1.1). The optical properties were measured using a UV-Vis-NIR spectrometer MPC3100UV-3100PC manufactured by Shimadzu Corporation under conditions of an optical path length: 1 cm and a measurement wavelength: 190 to 1300 nm. As a result of the SEM observation of the silver nanoplates in the aqueous suspension a, the silver nanoplates had an average particle diameter of 31 nm, an average thickness of 8 nm, and aspect ratios of 3.8. For the analysis of the SEM observation image, a scanning electron microscope SU-70 Manufactured by Hitachi, Ltd. was used.

### (3) Preparation of Gold-Coated Silver Nanoplates

To 120 mL of the aqueous suspension of silver nanoplates prepared in (2), 9.1 mL of an aqueous solution of 0.125 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold (hereinafter, suspension A1).

The main dispersion medium of the suspension A1 was water.

The gold-coated silver nanoplates contained in the suspension A1 were a mixture of plates having circular shapes and polygonal shapes including triangular shapes in which an average maximum length (particle diameter) of the main surfaces was 31 nm, and had an average thickness of 8 nm. Moreover, an average thickness of gold on the gold-coated silver nanoplates contained in the suspension A1 was 0.30 nm.

The suspension A1 had a polystyrenesulfonic acid (corresponding to the water-soluble polymer in the present invention) concentration of 0.98 nM.

The suspension A1 had a PVP (corresponding to the water-soluble polymer in the present invention) concentration of 8.1 μM. Note that the average thickness of gold was determined by selecting any ten gold-coated nanoplate particles from a HAADF-STEM image, measuring any ten sites on each of the particles to obtain data on thicknesses of gold at 100 sites in total, and excluding the highest and lowest 10% of the data to thus take the average of 80 sites for use as the average thickness of gold (the same shall also apply to suspensions B1 and D1 to be described later).

The suspension A1 had a pH of 4.0 at room temperature (20° C.). For the pH measurement, a twin pH meter B-212 manufactured by HORIBA, Ltd. was used (glass electrode method) (hereinafter the same).

The suspension A1 had a silver content of 0.0016% by mass relative to the total mass of the suspension.

### (4) pH Adjustment of Suspension A1

To 1.95 mL of the suspension A1 obtained in (3), 0.05 mL of a 200 mM PBS buffer solution and 0.025 mL of an aqueous solution of 190 mM sodium carbonate were added with stirring. Thus, a pH-adjusted suspension of gold-coated nanoplates was prepared (hereinafter, suspension B1).

The main dispersion medium of the suspension B1 was water.

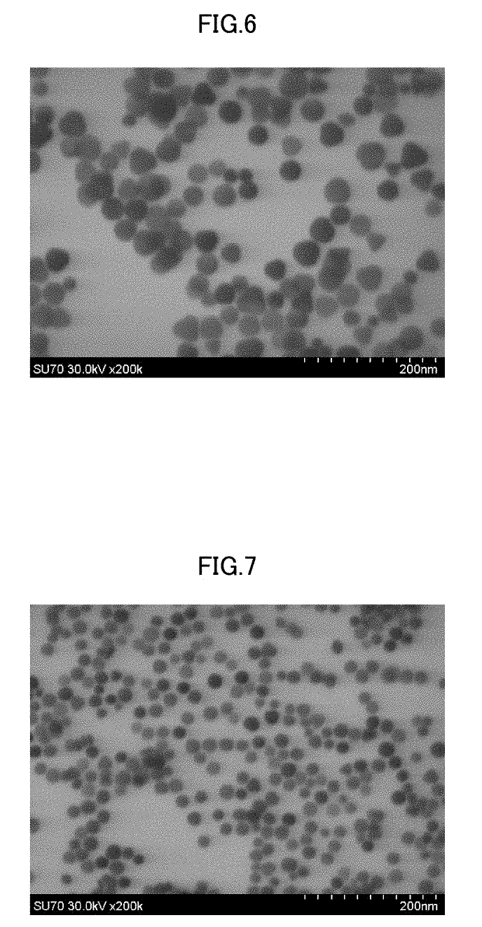

The gold-coated silver nanoplates contained in the suspension B1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension B1 was 0.30 nm. FIG. 6 shows a SEM observation image thereof. For the analysis of the SEM observation image, a scanning electron microscope SU-70 manufactured by Hitachi, Ltd. was used.

The color tone of a 3-fold diluted solution of the suspension B1 was magenta.

The chromaticity coordinates of the suspension B1 were x=0.4276 and y=0.1751. FIG. 1 shows the chromaticity coordinates in the CIE 1931 xy chromaticity diagram.

The chromaticity coordinates were measured using a UV-Vis-NIR spectrometer MPC3100UV-3100PC manufactured by Shimadzu Corporation under conditions of an optical path length: 1 cm, and an illumination: D65 and a field of view: 2° set in spectrometer operating software “Color Measurement Software (manufactured by Shimadzu Corporation).”

The suspension B1 had a polystyrenesulfonic acid concentration of 0.94 nM.

The suspension B1 had a PVP concentration of 7.8 μM.

The suspension B1 had a pH of 7.3 at room temperature (20° C.).

The suspension B1 had a silver content of 0.0015% by mass relative to the total mass of the suspension.

### Example 2

The suspension A1 (pH: 4.0) prepared in (3) of Example 1 was adjusted to have a pH of 5.2 by using an aqueous solution of 190 mM sodium carbonate in the same manner as in (4) of Example 1. Thus, a suspension F1 was prepared. The suspension F1 had a polystyrenesulfonic acid concentration of 0.94 nM. The suspension F1 had a PVP concentration of 7.8 M.

### Example 3

The suspension A1 (pH: 4.0) prepared in (3) of Example 1 was adjusted to have a pH of 9.8 by using an aqueous solution of 190 mM sodium carbonate in the same manner as in (4) of Example 1. Thus, a suspension G1 was prepared. The suspension G1 had a polystyrenesulfonic acid concentration of 0.94 nM. The suspension G1 had a PVP concentration of 7.8 μM.

### Example 4

To 120 mL of the aqueous suspension of silver nanoplates prepared in (2) of Example 1, 9.1 mL of ultrapure water and 9.6 mL of an aqueous solution of 0.14 mM chloroauric acid were added with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.99 mL of the prepared aqueous suspension of gold-coated silver nanoplates, 0.01 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension I1).

The main dispersion medium of the suspension I1 was water.

The gold-coated silver nanoplates contained in the suspension I1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension I1 was 0.30 nm.

The color tone of a 3-fold diluted solution of the suspension I1 was magenta.

The suspension I1 had a polystyrenesulfonic acid concentration of 0.94 nM.

The suspension I1 had a pH of 7.8 at room temperature (20° C.)

The suspension I1 had a silver content of 0.0016% by mass relative to the total mass of the suspension.

### Example 5

To 120 mL of the aqueous suspension of silver nanoplates prepared in (2) of Example 1, 9.1 mL of an aqueous solution of 0.780 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension J1).

The main dispersion medium of the suspension J1 was water.

The gold-coated silver nanoplates contained in the suspension J1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension J1 was 0.30 nm.

The color tone of a 3-fold diluted solution of the suspension J1 was magenta.

The suspension J1 had a polystyrenesulfonic acid concentration of 0.94 nM.

The suspension J1 had a PVP concentration of 49 μM.

The suspension J1 had a pH of 7.3 at room temperature (20° C.).

The suspension J1 had a silver content of 0.0015% by mass relative to the total mass of the suspension.

### Example 6

To 120 mL of the aqueous suspension of silver nanoplates prepared in (2) of Example 1, 9.1 mL of an aqueous solution of 0.025 mM SUNBRIGHT ME-020SH (molecular weight: 2,000, manufactured by NOF CORPORATION), which is a polyethylene glycol having an end modified with a thiol group, was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension K1).

The main dispersion medium of the suspension K1 was water. The gold-coated silver nanoplates contained in the suspension K1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension K1 Was 0.30 nm.

The color tone of a 3-fold diluted solution of the suspension K1 was magenta.

The suspension K1 had a polystyrenesulfonic acid concentration of 0.94 nM.

The suspension K1 had a SUNBRIGHT ME-020SH concentration of 1.6 μM.

The suspension K1 had a pH of 7.3 at room temperature (20° C.)

The suspension K1 had a silver content of 0.0015% by mass relative to the total mass of the suspension.

### Example 7

### Preparation of Silver Nanoplates (Color Tone: Yellow)

To 200 mL of ultrapure water, 4.5 mL of an aqueous solution of 10 mM ascorbic acid was added, and further 12 mL of the aqueous suspension of silver-nanoplate seed particles prepared in (1) of Example 1 was added. To the obtained solution, 120 mL of an aqueous solution of 0.5 mM silver nitrate was added with stirring at 30 mL/min. The stirring was stopped four minutes after the addition of the aqueous solution of silver nitrate was completed. Then, 20 mL of an aqueous solution of 25 mM sodium citrate was added thereto. The obtained solution was left standing in an incubator (30° C.) in an air atmosphere for 100 hours. Thereby, an aqueous suspension b of silver nanoplates was prepared. The prepared suspension was 4-fold diluted by volume with ultrapure water. FIG. 5 shows optical properties of the aqueous suspension. The maximum absorption was exhibited at a wavelength of 454 nm (extinction 1.1). The optical properties were measured using a UV-Vis-NIR spectrometer MPC3100UV-3100PC manufactured by Shimadzu Corporation under conditions of an optical path length: 1 cm and a measurement wavelength: 190 to 1300 nm. As a result of the SEM observation of the silver nanoplates in the aqueous suspension b, the silver nanoplates had an average particle diameter of 18 nm, an average thickness of 8 nm, and aspect ratios of 2.2. For the analysis of the SEM observation image, a scanning electron microscope SU-70 manufactured by Hitachi, Ltd. was used.

### Preparation of Gold-Coated Silver Nanoplates

To 120 mL of the aqueous suspension of silver nanoplates prepared as described above, 9.1 mL of an aqueous solution of 0.125 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold (hereinafter, suspension M1).

The main dispersion medium of the suspension M1 was water.

The gold-coated silver nanoplates contained in the suspension M1 were a mixture of plates having circular shapes and polygonal shapes including triangular shapes in which an average maximum length (particle diameter) of the main surfaces was 18 nm, and had an average thickness of 8 nm. Moreover, an average thickness of gold on the gold-coated silver nanoplates contained in the suspension M1 was 0.30 nm.

The suspension M1 had a polystyrenesulfonic acid (corresponding to the water-soluble polymer in the present invention) concentration of 0.98 nM.

The suspension M1 had a pH of 4.0 at room temperature (20° C.). For the pH measurement, a twin pH meter B-212 manufactured by HORIBA, Ltd. was used (glass electrode method) (hereinafter the same).

The suspension M1 had a silver content of 0.0016% by mass relative to the total mass of the suspension.

### pH Adjustment of Suspension M1

To 1.95 mL of the suspension M1, 0.05 mL of a 200 mM PBS buffer solution and 0.025 mL of an aqueous solution of 190 mM sodium carbonate were added with stirring. Thus, a pH-adjusted suspension of gold-coated nanoplates was prepared (hereinafter, suspension N1).

The main dispersion medium of the suspension N1 was water.

The gold-coated silver nanoplates contained in the suspension N1 were a mixture of plates having circular shapes and polygonal shapes including triangular shapes in which an average maximum length of the main surfaces was 18 nm, and had an average thickness of 8 nm. Moreover, an average thickness of gold on the gold-coated silver nanoplates contained in the suspension N1 was 0.30 nm.

FIG. 7 shows a SEM Observation image thereof. For the analysis of the SEM observation image, a scanning electron microscope SU-70 manufactured by Hitachi, Ltd. was used.

The color tone of a 3-fold diluted solution of the suspension N1 was yellow.

The chromaticity coordinates of the suspension N1 were x=0.5070 and y=0.4774. FIG. 1 shows the chromaticity coordinates in the CIE 1931 xy chromaticity diagram.

Meanwhile, the color tone of a mixed solution (B1+N1) obtained by mixing the suspensions B1 and N1 together was red.

The chromaticity coordinates of the mixed solution (B1+N1) were x=0.6057 and v=0.3317.

FIG. 0520-2 shows the chromaticity coordinates in the CIE 1931 xy chromaticity diagram.

The suspension N1 had a polystyrenesulfonic acid concentration of 0.94 nM.

The suspension N1 had a PVP concentration of 7.8 M.

The suspension N1 had a pH of 7.3 at room temperature (20° C.)

The suspension N1 had a silver content of 0.0015% by mass relative to the total mass of the suspension.

The suspension N1 was prepared as Example 7.

### Example 8

### (1) Preparation of Silver Nanoplates (Color Tone: Cyan)

To 200 mL of ultrapure water, 4.5 mL of an aqueous solution of 10 mM ascorbic acid was added, and further 2 mL of the aqueous suspension of silver-nanoplate seed particles prepared in (1) of Example 1 was added. To the obtained solution, 120 mL of an aqueous solution of 0.5 mM silver nitrate was added with stirring at 30 mL/min. The stirring was stopped four minutes after the addition of the aqueous solution of silver nitrate was completed. Then, 20 mL of an aqueous solution of 25 mM sodium citrate was added thereto. The obtained solution was left standing in an incubator (30° C.) in an air atmosphere for 100 hours. Thereby, an aqueous suspension c of silver nanoplates was prepared. The prepared suspension was 4-fold diluted by volume with ultrapure water. FIG. 5 shows optical properties of the aqueous suspension. The maximum absorption was exhibited at a wavelength of 626 nm (extinction 1.1). The optical properties were measured using a UV-Vis-NIR spectrometer MPC3100UV-3100PC manufactured by Shimadzu Corporation under conditions of an optical path length: 1 cm and a measurement wavelength: 190 to 1300 nm. As a result of the SEM observation of the silver nanoplates in the aqueous suspension c, the silver nanoplates had an average particle diameter of 50 nm, an average thickness of 10 nm, and aspect ratios of 5.0. For the analysis of the SEM observation image, a scanning electron microscope SU-70 manufactured by Hitachi, Ltd. was used.

### (2) Preparation of Gold-Coated Silver Nanoplates

To 120 mL of the aqueous suspension of silver nanoplates prepared as described above, 9.1 mL of an aqueous solution of 0.125 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold (hereinafter, suspension O1).

The main dispersion medium of the suspension O1 was water.

The gold-coated silver nanoplates contained in the suspension O1 were a mixture of plates having circular shapes and polygonal shapes including triangular shapes in which an average maximum length (particle diameter) of the main surfaces was 50 nm, and had an average thickness of 10 nm. Moreover, an average thickness of gold on the gold-coated silver nanoplates contained in the suspension O1 was 0.30 nm.

The color tone of a 3-fold diluted solution of the suspension O1 was cyan.

The suspension O1 had a polystyrenesulfonic acid (corresponding to the water-soluble polymer in the present invention) concentration of 0.98 nM.

The suspension O1 had a pH of 4.0 at room temperature (20° C.). For the pH measurement, a twin pH meter B-212 manufactured by HORIBA, Ltd. was used (glass electrode method) (hereinafter the same).

The suspension O1 had a silver content of 0.0016% by mass relative to the total mass of the suspension.

### (3) pH Adjustment of Suspension O1

To 1.95 mL of the suspension O1, 0.05 mL of a 200 mM PBS buffer solution and 0.025 mL of an aqueous solution of 190 mM sodium carbonate were added with stirring. Thus, a pH-adjusted suspension of gold-coated nanoplates was prepared (hereinafter, suspension P1).

The main dispersion medium of the suspension P1 was water.

The gold-coated silver nanoplates contained in the suspension P1 were a mixture of plates having circular shapes and polygonal shapes including triangular shapes in which an average maximum length of the main surfaces was 50 nm, and had an average thickness of 10 nm. Moreover, an average thickness of gold on the gold-coated silver nanoplates contained in the suspension P1 was 0.30 nm.

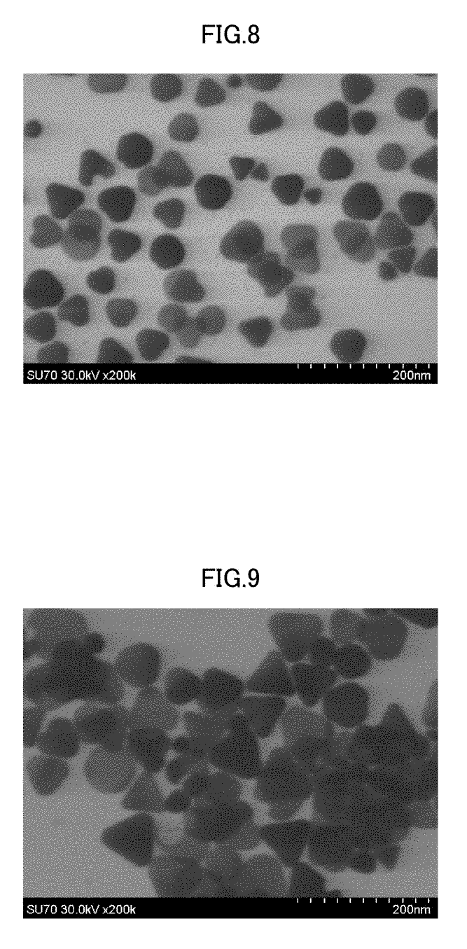

FIG. 8 shows a SEM observation image thereof. For the analysis of the SEM observation image, a scanning electron microscope SU-70 manufactured by Hitachi, Ltd. was used.

The color tone of a 3-fold diluted solution of the suspension P1 was cyan.

The chromaticity coordinates of the suspension P1 were x=0.1467 and y=0.2090. FIG. 1 shows the chromaticity coordinates in the CIE 1931 xy chromaticity diagram.

Meanwhile, the color tone of a mixed solution (B1+P1) obtained by mixing the suspensions 51 and P1 together was blue, while the color tone of a mixed solution (N1+P1) obtained by mixing the suspensions N1 and P1 together was green.

The chromaticity coordinates of the mixed solution (B1+P1) were x=0.1731 and y=0.0675. The chromaticity coordinates of the mixed solution (N1+P1) were x=0.2549 and y=0.5712. FIG. 2 shows the chromaticity coordinates in the CIE 1931 xy chromaticity diagram.

The suspension P1 had a polystyrenesulfonic acid concentration of 0.94 nM.

The suspension P1 had a PVP concentration of 7.8 μM.

The suspension P1 had a pH of 7.3 at room temperature (20° C.)

The suspension P1 had a silver content of 0.0015% by mass relative to the total mass of the suspension.

### Example 9

### (1) Preparation of Silver Nanoplates (Color Tone: Light Cyan)

To 200 mL of ultrapure water, 4.5 mL of an aqueous solution of 10 mM ascorbic acid was added, and further 1 mL of the aqueous suspension of silver-nanoplate seed particles prepared in (1) of Example 1 was added. To the obtained solution, 120 mL of an aqueous solution of 0.5 mM silver nitrate was added with stirring at 30 mL/min. The stirring was stopped four minutes after the addition of the aqueous solution of silver nitrate was completed. Then, 20 mL of an aqueous solution of 25 mM sodium citrate was added thereto. The obtained solution was left standing in an incubator (30° C.) in an air atmosphere for 100 hours. Thereby, an aqueous suspension d of silver nanoplates was prepared. The prepared suspension was 4-fold diluted by volume with ultrapure water. FIG. 5 shows optical properties of the aqueous suspension. The maximum absorption was exhibited at a wavelength of 704 nm (extinction 1.0). The optical properties were measured using a UV-Vis-NTR spectrometer MPC3100UV-3100PC manufactured by Shimadzu Corporation under conditions of an optical path length: 1 cm and a measurement wavelength: 190 to 1300 nm. As a result of the SEM observation of the silver nanoplates in the aqueous suspension d, the silver nanoplates had an average particle diameter of 74 nm, an average thickness of 8 nm, and aspect ratios of 9.2. For the analysis of the SEM observation image, a scanning electron microscope SU-70 manufactured by Hitachi, Ltd. was used.

### (2) Preparation of Gold-Coated Silver Nanoplates

To 120 mL of the aqueous suspension of silver nanoplates prepared as described above, 9.1 mL of an aqueous solution of 0.125 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold (hereinafter, suspension Q1).

The main dispersion medium of the suspension Q1 was water.

The gold-coated silver nanoplates contained in the suspension Q1 were a mixture of plates having circular shapes and polygonal shapes including triangular shapes in which an average maximum length (particle diameter) of the main surfaces was 74 nm, and had an average thickness of 8 nm. Moreover, an average thickness of gold on the gold-coated silver nanoplates contained in the suspension Q1 was 0.30 nm.

FIG. 9 shows a SEM observation image thereof. For the analysis of the SEM observation image, a scanning electron microscope SU-70 manufactured by Hitachi, Ltd. was used.

The color tone of a 3-fold diluted solution of the suspension Q1 was light cyan.

The suspension Q1 had a polystyrenesulfonic acid (corresponding to the water-soluble polymer in the present invention) concentration of 0.98 nM.

The suspension Q1 had a pH of 4.0 at room temperature (20° C.). For the pH measurement, a twin pH meter B-212 manufactured by HORIBA, Ltd. was used (glass electrode method) (hereinafter the same).

The suspension Q1 had a silver content of 0.0016% by mass relative to the total mass of the suspension.

### (3) pH Adjustment of Suspension Q1

To 1.95 mL of the suspension Q1, 0.05 mL of a 200 mM PBS buffer solution and 0.025 mL of an aqueous solution of 190 mM sodium carbonate were added with stirring. Thus, a pH-adjusted suspension of gold-coated nanoplates was prepared (hereinafter, suspension R1).

The main dispersion medium of the suspension R1 was water.

The gold-coated silver nanoplates contained in the suspension R1 were a mixture of plates having circular shapes and polygonal shapes including triangular shapes in which an average maximum length of the main surfaces was 74 nm, and had an average thickness of 8 nm. Moreover, an average thickness of gold on the gold-coated silver nanoplates contained in the suspension R1 was 0.30 nm.

The color tone of a 3-fold diluted solution of the suspension R1 was light cyan.

The suspension R1 had a polystyrenesulfonic acid concentration of 0.94 nM.

The suspension R1 had a PVP concentration of 7.8 μM.

The suspension R1 had a pH of 7.3 at room temperature (20° C.).

The suspension R1 had a silver content of 0.0015% by mass relative to the total mass of the suspension.

### Example 10

### (1) Preparation of Gold-Coated Silver Nanoplates

An aqueous suspension of gold-coated silver nanoplates (hereinafter, suspension S1) was prepared in the same manner as in the preparation of the suspension A1, except that the concentration of chloroauric acid aqueous solution in the preparation of the gold-coated silver nanoplates described above in (3) of Example 1 was changed from 0.14 mM to 0.42 mM.

The main dispersion medium of the suspension S1 was water.

The gold-coated silver nanoplates contained in the suspension 51 were a mixture of plates having circular shapes and polygonal shapes including triangular shapes in which an average maximum length (particle diameter) of the main surfaces was 31 nm, and had an average thickness of 8 nm. Moreover, an average thickness of gold on the gold-coated silver nanoplates contained in the suspension S1 was 0.85 nm.

The suspension S1 bad a polystyrenesulfonic acid concentration of 0.98 nM.

The suspension S1 had a PVP concentration of 8.1 μM. Note that the average thickness of gold was determined by selecting any ten gold-coated nanoplate particles from a HAADF-STEM image, measuring any ten sites on each of the particles to obtain data on thicknesses of gold at 100 sites in total, and excluding the highest and lowest 10% of the data to thus take the average of 80 sites for use as the average thickness of gold.

The suspension S1 had a pH of 4.0 at room temperature (20° C.). For the pH measurement, a twin pH meter B-212 manufactured by HORIBA, Ltd. was used (glass electrode method).

The suspension S1 had a silver content of 0.0016% by mass relative to the total mass of the suspension.

### (2) pH Adjustment of Suspension S1

To 1.95 mL of the suspension S1 obtained in (1), 0.05 mL of a 200 mM PBS buffer solution and 0.025 mL of an aqueous solution of 190 mM sodium carbonate were added with stirring. Thus, a pH-adjusted suspension of gold-coated nanoplates was prepared (hereinafter, suspension T1).

The main dispersion medium of the suspension T1 was water.

The gold-coated silver nanoplates contained in the suspension T1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension T1 was 0.90 nm.

The color tone of a 3-fold diluted solution of the suspension T1 was magenta.

The suspension T1 had a polystyrenesulfonic acid concentration of 0.94 nM.

The suspension T1 had a PVP concentration of 7.8 μM.

The suspension T1 had a pH of 7.4 at room temperature (20° C.).

The suspension T1 had a silver content of 0.0015% by mass relative to the total mass of the suspension.

### COMPARATIVE EXAMPLES

(1) Example where Water-Soluble Polymer Concentration is High (Part 1)

To 120 mL of the aqueous suspension of silver nanoplates prepared in (2) of Example 1, 9.1 mL of an aqueous solution of 1.25 mM polyvinylpyrrolidone (PVP) (molecular weight: 40,000) was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension D1).

The main dispersion medium of the suspension D1 was water.

The gold-coated silver nanoplates contained in the suspension D1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension D1 was 0.30 nm.

The color tone of a 3-fold diluted solution of the suspension D1 was magenta.

The suspension D1 had a polystyrenesulfonic acid concentration of 0.94 nM.

The suspension D1 had a PVP concentration of 78 μM.

The suspension D1 had a pH of 7.3 at room temperature (20° C.) The suspension D1 had a silver content of 0.0015% by mass relative to the total mass of the suspension.

(2) Example where pH is High

The suspension A1 (pH: 4.0) prepared in (3) of Example 1 was adjusted to have a pH of 11.5 by using an aqueous solution of 200 mM sodium hydroxide in the same manner as in (4) of Example 1. Thus, a suspension H1 was prepared. The suspension H1 had a polystyrenesulfonic acid concentration of 0.94 nM. The suspension H1 had a PVP concentration of 7.8 μM.

(3) Example where Water-Soluble Polymer Concentration is High (Part 2)

To 120 mL of the aqueous suspension of silver nanoplates prepared in (2) of Example 1, 9.1 mL of an aqueous solution of 1.25 mM SUNBRIGHT ME-020SH (molecular weight: 2,000, manufactured by NOF CORPORATION), which is a polyethylene glycol having an end modified with a thiol group, was added, and 1.6 mL of an aqueous solution of 0.5 M ascorbic acid was added. Then, 9.6 mL of an aqueous solution of 0.14 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension L1).

The main dispersion medium of the suspension L1 was water.

The gold-coated silver nanoplates contained in the suspension L1 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension L1 was 0.30 nm.

The color tone of a 3-fold diluted solution of the suspension L1 was magenta.

The suspension L1 had a polystyrenesulfonic acid concentration of 0.94 nM.

The suspension L1 had a SUNBRIGHT ME-020SH concentration of 78 μM.

The suspension L1 had a pH of 7.3 at room temperature (20° C.)

The suspension L1 had a silver content of 0.0015% by mass relative to the total mass of the suspension.

As described above, the suspensions of gold-coated silver nanoplates of the present invention and the suspensions of Comparative Examples were prepared. Table 2 to be shown later summarizes the concentration of the water-soluble polymer (polystyrenesulfonic acid (PSS), polyvinylpyrrolidone (PVP), or thiol-modified polyethylene glycol (PEG-SH)) in the suspension A1, B1, F1, G1, I1, J1, K1, N1, P1, D1, H1, L1, or T1, as well as the pH and the color tone (magenta (M), yellow (Y), cyan (C), or light cyan (PC)) thereof.

### Experiment 1

Each of the suspensions of Examples and Comparative Examples was subjected to an immunochromatographic test according to the following procedure. The test result was evaluated.

(1) Preparation of Developing Solutions Used in Immunochromatographic Test (Supporting of Specific Binding Substance onto Gold-Coated Silver Nanoplates (Labeling of Detection Reagent))

First, 0.2 mL of a solution of an antibody (product name: Goat anti HBsAg, manufactured by: Arista Biologicals, Inc.) (detection antibody in the immunochromatography) against a hepatitis B virus antigen (HBs antigen) at a concentration of 50 μg/mL in a 5 mM PBS(−) buffer solution was mixed with 1.8 mL of the suspension of gold-coated silver nanoplates (the suspension A1, B1, F1, G1, I1, J1, J1, N1, P1, D1, H1, L1, or T1). The obtained mixture was shaken at room temperature for 30 minutes. Then, the resultant was centrifuged (25000 rpm, 4° C., 10 minutes) to precipitate a complex of the antibody and the gold-coated silver nanoplate, and 1.85 mL of the supernatant was removed. Subsequently, the complex of the antibody and the gold-coated silver nanoplates was dispersed again by adding 1.85 mL of a 5 mM PBS (−) buffer solution containing 4.9 μM BSA. The resultant was adjusted to have an extinction of 2.0 by using a UV visible spectrometer Agilent 8453 (manufactured by Agilent Technologies, Inc.). In this manner, developing solutions were prepared.

Table 1 below shows relations between the suspensions of gold-coated silver nanoplates used and the prepared developing solutions.

TABLE 1
Relations between suspensions of gold-coated silver
nanoplates and prepared developing solutions

| Suspension of gold-coated | |
|---|---|
| silver nanoplates | Developing solution |
| Suspension A1 | Developing solution A1 |
| Suspension B1 | Developing solution B1 |
| Suspension F1 | Developing solution F1 |
| Suspension G1 | Developing solution G1 |
| Suspension I1 | Developing solution I1 |
| Suspension J1 | Developing solution J1 |
| Suspension K1 | Developing solution K1 |
| Suspension N1 | Developing solution N1 |
| Suspension P1 | Developing solution P1 |
| Suspension R1 | Developing solution R1 |
| Suspension T1 | Developing solution T1 |
| Suspension D1 | Developing solution D1 |
| Suspension H1 | Developing solution H1 |
| Suspension L1 | Developing solution L1 |

### (2) Immunochromatographic Test

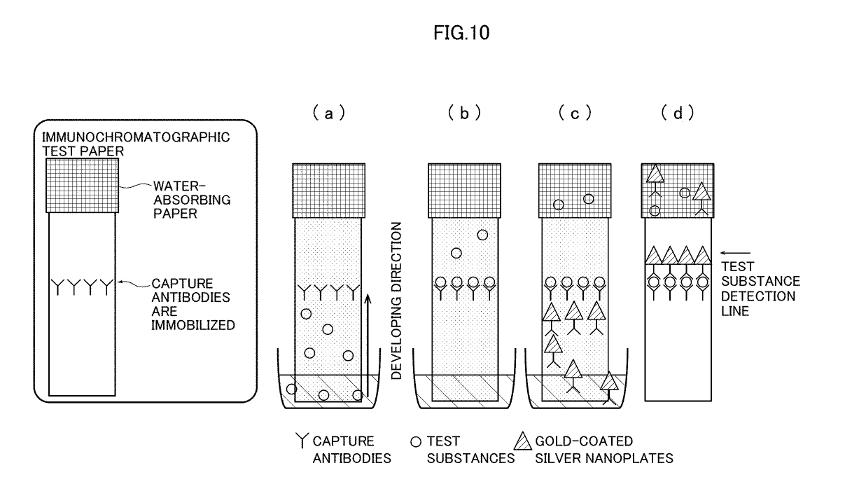

According to the procedure illustrated in FIG. 10, an immunochromatographic test with the test substance of a hepatitis B virus antigen (HBs antigen) was conducted using an immunochromatographic test paper having an anti-HBs antigen antibody (capture antibody) immobilized in a straight line (the detection line of FIG. 10).

The immunochromatographic test paper used was purchased from a contractor company (BioDevice Technology, Co., Ltd.) for preparing immunochromatographic test papers. When the capture antibody was immobilized in a straight line, the capture antibody used was adjusted to a concentration of 1 g/mL by using a 5 mM PBS (−) buffer solution.

A first developing solution (developing solution used in FIG. 10(a)) was a solution of an HBs antigen (product name: HBsAg Protein (Subtype adr), manufactured by: Fitzgerald Industries International Inc.) in a 5 mM PBS(−) buffer solution. As the first developing solution, solutions were prepared which had HBs antigen concentrations of 0.60 μM, 0.06 μM, 0.006 μM, 0.0006 μM, and 0 M (blank), respectively.

A second developing solution was a 5 mM PBS(−) buffer solution.

A third developing solution (developing solution used in FIG. 10(c)) was the above-described developing solution A1, B1, F1, G1, I1, J1, K1, N1, P1, D1, H1, L1, or T1.

The specific test procedure was as follows.

On the immunochromatographic test paper, 15 μL of the first developing solution was developed (FIG. 10(a)). As the first developing solution was developed, the HBs antigen was captured by the capture antibody immobilized on the detection line of the test paper (FIG. 10(b)).

Then, 30 μL of the second developing solution was developed to wash away excessive antigens on the immunochromatographic test paper.

Finally, 60 μL of the third developing solution was developed (FIG. 10(c)). As the third developing solution was developed, the detection antibody (the complex of the antibody and the gold-coated silver nanoplates) bound to the HBs antigen captured on the detection line of the test paper (FIG. 10(d)).

The same procedure was repeated using each of the first developing solutions having different HBs concentrations.

### (3) Immunochromatographic Test Evaluation by Visual Judgment

After the third developing solution was developed, the coloring of the gold-coated silver nanoplates on the detection line (the color tone of the suspension; to be more specific, magenta when the developing solution B1 was used, yellow when the developing solution N1 was used, cyan when the developing solution P1 was used) was visually checked to judge the presence or absence of the HBs antigen. Table 2 below shows the result.

### (4) Immunochromatographic Test Evaluation by Luminance Analysis

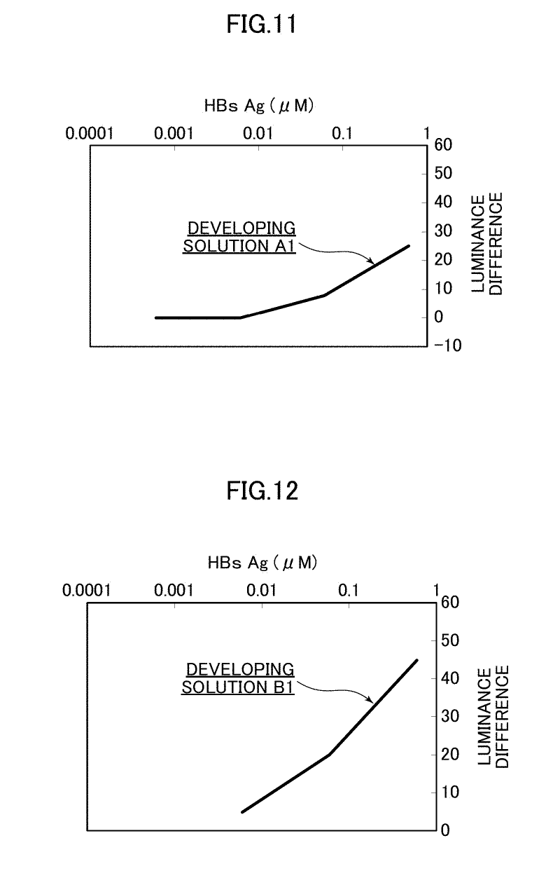

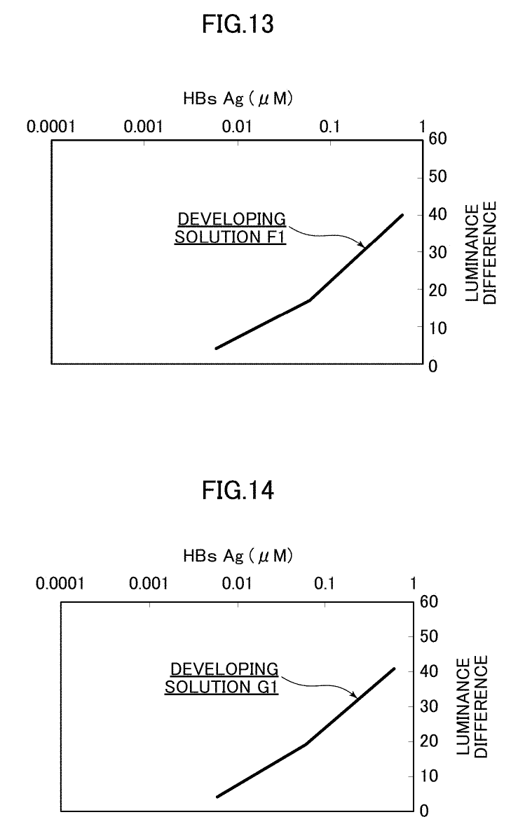

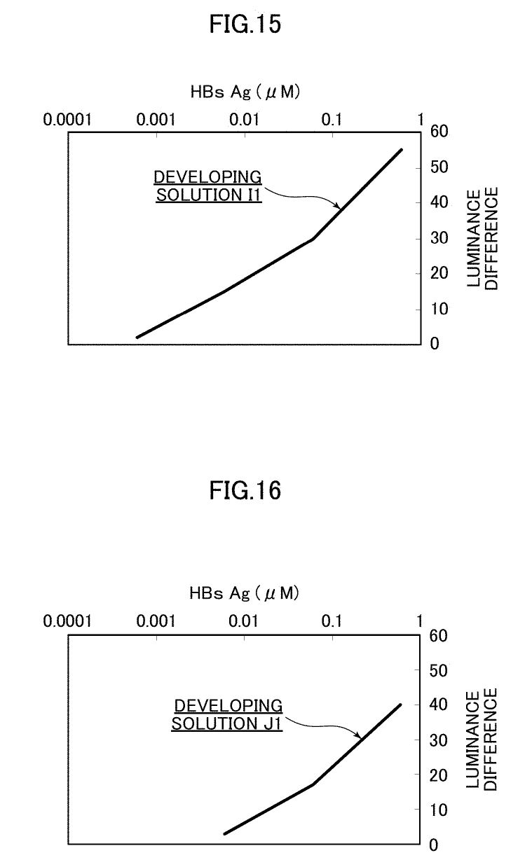

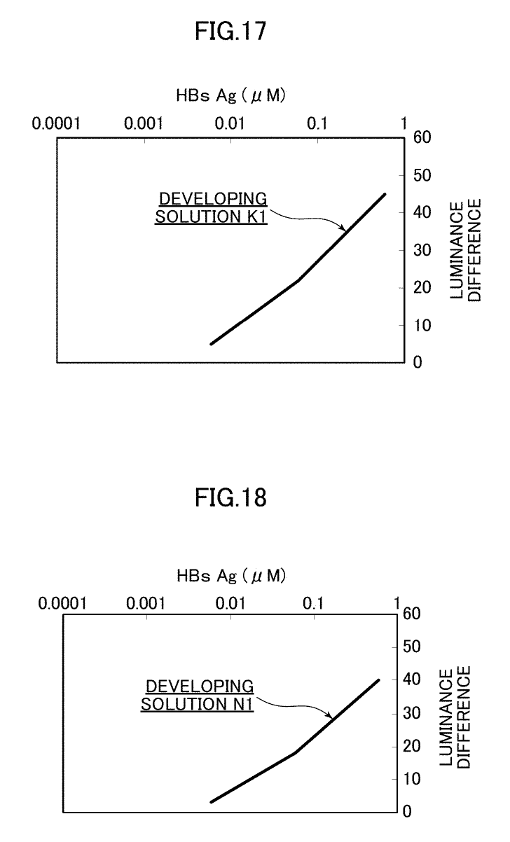

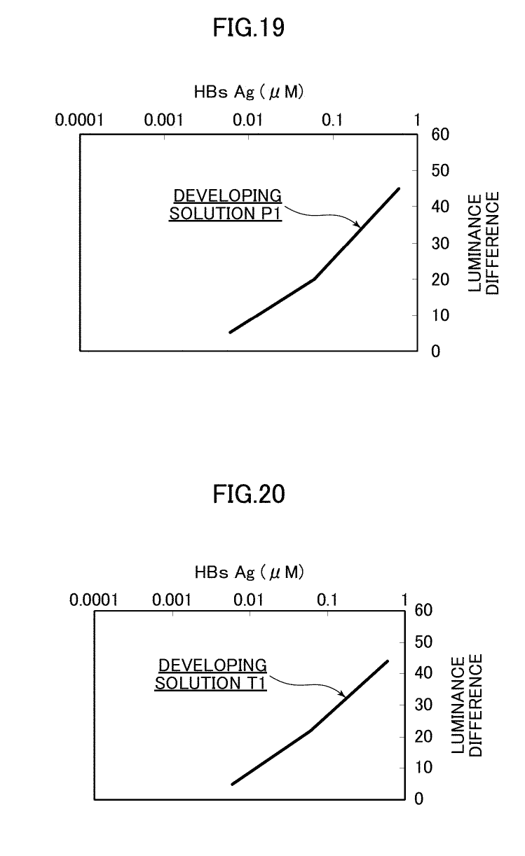

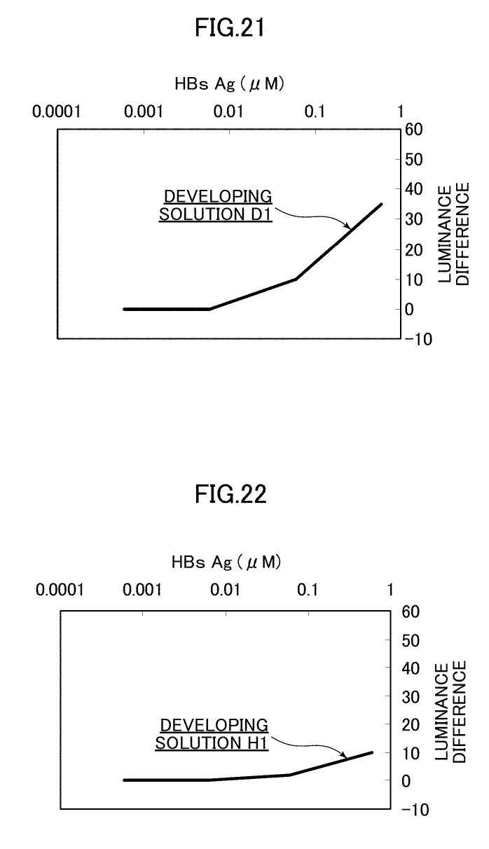

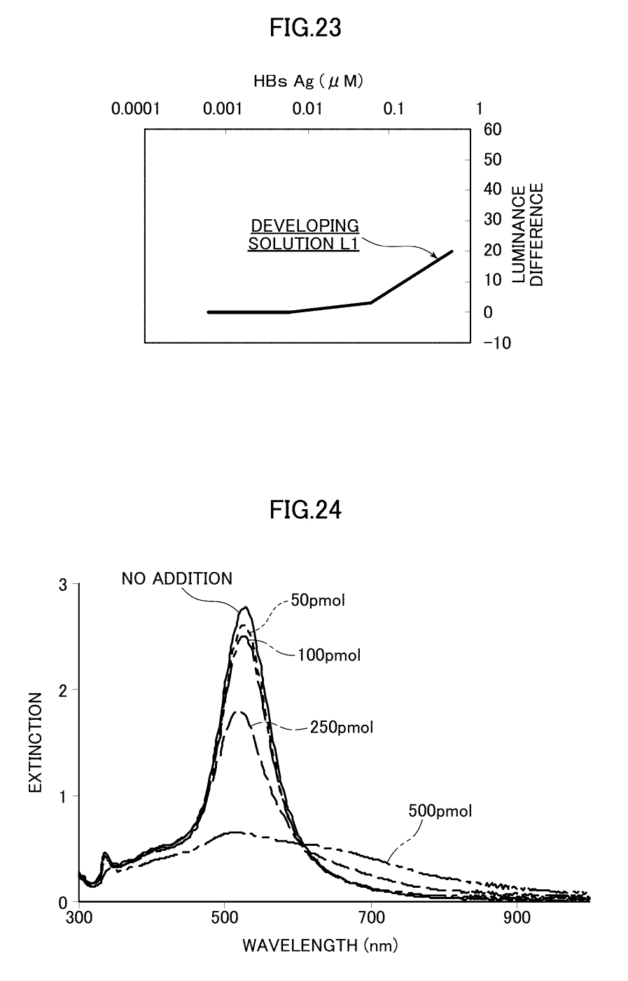

After the third developing solution was developed, the immunochromatographic test paper was scanned (apparatus name: Cano Scan LiDE500F, manufactured by: Canon Inc.) to quantify the lowest luminance at the detection line (immobilized section of the capture antibody) and the lowest luminance at a section other than the detection line by using image analysis software (Image-J: open-source, public-domain image processing software (http://imagej.nih.gov/ij/) developed by Wayne Rasband at the National Institutes of Health). The lowest luminance was determined by measuring five times luminances at different positions in the target region (the detection line or the section other than the detection line) and adopting a median of the obtained numerical values as the lowest luminance. The luminance difference was calculated according to (the lowest luminance at the detection line−the lowest luminance at the section other than the detection line). The following Table 2 and FIGS. 11 to 23 show the result.

TABLE 2
Properties of suspensions used in the immunochromatography and test results
<Properties of Suspensions>

| Suspension | A1 | B1 | F1 | G1 | I1 | J1 | K1 | N1 | P1 | R1 | T1 | D1 | H1 | L1 |
|---|---|---|---|---|---|---|---|---|---|---|---|---|---|---|
| PSS (nM) | 0.98 | 0.94 | 0.94 | 0.94 | 0.94 | 0.94 | 0.94 | 0.94 | 0.94 | 0.94 | 0.94 | 0.94 | 0.94 | 0.94 |
| PVP (μM) | 8.1 | 7.8 | 7.8 | 7.8 | 0 | 49 | 0 | 7.8 | 7.8 | 7.8 | 7.8 | 78 | 7.8 | 0 |
| PEG-SH (μM) | 0 | 0 | 0 | 0 | 0 | 0 | 1.6 | 0 | 0 | 0 | 0 | 0 | 0 | 78 |
| pH | 4.0 | 7.3 | 5.2 | 9.8 | 7.8 | 7.3 | 7.3 | 7.3 | 7.3 | 7.3 | 7.4 | 7.3 | 11.5 | 7.3 |
| color tone | M | M | M | M | M | M | M | Y | C | PC | M | M | M | M |

<HBs antigen concentration (μM) in the first developing solution and visual judgment result>

| Developing solution | | A1 | B1 | F1 | G1 | I1 | J1 | K1 | N1 | P1 | R1 | T1 | D1 | H1 | L1 |
|---|---|---|---|---|---|---|---|---|---|---|---|---|---|---|---|
| antigen | 0.6 | + | + | + | + | + | + | + | + | + | N/A | + | + | + | + |
| | 0.06 | + | + | + | + | + | + | + | + | + | N/A | + | + | + | + |
| | 0.006 | − | + | + | + | + | + | + | + | + | N/A | + | − | − | − |
| | 0.0006 | N/A | N/A | N/A | N/A | + | N/A | N/A | N/A | N/A | N/A | N/A | N/A | N/A | N/A |

| | | | | | | | | | | | | | | |
|---|---|---|---|---|---|---|---|---|---|---|---|---|---|---|
| 0 (blank) | − | − | − | − | − | − | − | − | − | N/A | − | − | − | − |

+: the coloring on the detection line was observed.
−: the coloring on the detection line was not observed.
N/A: no test data available.

<HBs antigen concentration (μM) in the first developing solution and luminance analysis result>

| Developing solution | | A1 | B1 | F1 | G1 | I1 | J1 | K1 | N1 | P1 | R1 | T1 | D1 | H1 | L1 |
|---|---|---|---|---|---|---|---|---|---|---|---|---|---|---|---|
| antigen | 0.6 | 25 | 45 | 40 | 41 | 60 | 40 | 45 | 40 | 45 | N/A | 44 | 35 | 10 | 20 |
| | 0.06 | 8 | 20 | 17 | 19 | 40 | 17 | 22 | 18 | 20 | N/A | 22 | 10 | 2 | 2 |
| | 0.006 | 0 | 5 | 4 | 4 | 17 | 3 | 5 | 3 | 5 | N/A | 5 | 0 | 0 | 0 |
| | 0.0006 | N/A | N/A | N/A | N/A | 2 | N/A | N/A | N/A | N/A | N/A | N/A | N/A | N/A | N/A |

| | | | | | | | | | | | | | | |
|---|---|---|---|---|---|---|---|---|---|---|---|---|---|---|
| 0 (blank) | 0 | 0 | 0 | 0 | 0 | 0 | 0 | 0 | 0 | N/A | 0 | 0 | 0 | 0 |

Numerical values in the column of each developing solution indicate luminance differences.
N/A: no test data available.

Even when the suspension E1 of gold-coated silver nanoplates supporting no antibody was used as the third developing solution, the color of the detection line did not change, and no luminance difference was found, either. The following Table 3 shows the result.

TABLE 3
Test result when the suspension B1 of gold-coated
silver nanoplates supporting no antibody was used as the
third developing solution

| HBs antigen | | |
|---|---|---|
| concentration (μM) | Visual judgment | Luminance analysis |
| 0.6 | − | 0 |
| 0.06 | − | 0 |
| 0.006 | − | 0 |
| 0.0006 | N/A | N/A |
| 0 (blank) | − | 0 |

+: the coloring on the detection line was observed.
−: the coloring on the detection line was not observed.
Numerical values in the column of the luminance analysis indicate luminance differences.
N/A: no test data available.

In contrast, when the suspension of gold-coated silver nanoplates supporting the anti-HBs antigen antibody was used as the third developing solution, the detection line was colored (the coloring occurred in the color tone of the suspension; to be more specific, magenta when the developing solution B1 was used, yellow when the developing solution N1 was used, cyan when the developing solution P1 was used), and a remarkable luminance difference was also measured. This is because the gold-coated silver nanoplates supporting the anti-HBs antigen antibody formed a complex with the HBs antigen captured by the capture antibody on the detection line.

Moreover, the developing solution prepared from the suspension A1, E1, F1, G1, I1, J1, K1, N1, P1, D1, L1, or T1 having a pH of 10 or less had a higher luminance difference than that of the developing solution prepared from the suspension H1 having a pH of 11.5. This revealed that the former has a higher detection sensitivity than the latter.

Further, the developing solution prepared from the suspension B1, F1, G1, I1, J1, K1, N1, P1, or T1 having a water-soluble polymer (PVP or PEG-SH) concentration of 50 μM or less had a higher luminance difference than that of the developing solution prepared from the suspension D1 or L1 having a water-soluble polymer concentration of 78 μM. This revealed that the former has a higher detection sensitivity than the latter. Particularly, the developing solution I1 prepared from the suspension I1 had a luminance difference even when 0.0006 μM of the HBs antigen was used.

### Example 11

### 1. Preparation of Fine Metal Particle Suspension

### 1-1. Preparation of Silver Nanoplates

### 1-1-1. Preparation of Silver-Nanoplate Seed Particles

To 20 mL of an aqueous solution of 2.5 mM sodium citrate, 1 mL of an aqueous solution of 0.5 g/L polystyrenesulfonic acid having a molecular weight of 70,000 and 1.2 mL of an aqueous solution of 10 mM sodium borohydride were added. Then, 50 mL of an aqueous solution of 0.5 mM silver nitrate was added thereto with stirring at 20 mL/min. The obtained solution was left standing in an incubator (30° C.) for 60 minutes. Thereby, an aqueous suspension of silver-nanoplate seed particles was prepared.

### 1-1-2. Preparation of Silver Nanoplate Suspension A2

To 200 ml of ultrapure water, 4.5 mL of an aqueous solution of 10 mM ascorbic acid was added, and 12 ml of the above-described aqueous suspension of silver-nanoplate seed particles was added. To the obtained solution, 120 mL of an aqueous solution of 0.5 mM silver nitrate was added with stirring at 30 mL/min. The stirring was stopped four minutes after the addition of the aqueous solution of silver nitrate was completed. Then, 20 ml of an aqueous solution of 25 mM sodium citrate was added thereto. The obtained solution was left standing in an incubator (30° C.) in an air atmosphere for 100 hours. Thereby, a silver nanoplate suspension A2 was prepared, which is an aqueous suspension of plate-shaped silver nanoparticles.

### 1-1-3. Preparation of Silver Nanoplate Suspension B2

A silver nanoplate suspension B2, which is an aqueous suspension of plate-shaped silver nanoparticles, was prepared in the same manner as the silver nanoplate suspension A2, except that the amount of the aqueous suspension of silver-nanoplate seed particles added was changed from 12 ml to 4 ml.

### 1-1-4. Preparation of Silver Nanoplate Suspension C2

A silver nanoplate suspension C2, which is an aqueous suspension of plate-shaped silver nanoparticles, was prepared in the same manner as in the preparation of the silver nanoplate suspension A2, except that the amount of the aqueous suspension of silver-nanoplate seed particles added was changed from 12 ml to 2 ml.

### 1-2. Preparation of Gold-Coated Silver Nanoplates

### 1-2-1. Preparation of Gold-Coated Silver Nanoplate Suspension A2

To 120 ml of the silver nanoplate suspension A2, 9.1 ml of an aqueous solution of 0.125 mM polyvinylpyrrolidone (PVP) (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, a gold-coated silver nanoplate suspension A2 was prepared.

The main dispersion medium of the gold-coated silver nanoplate suspension A2 was water.

As a result of the scanning electron microscope (SEM) observation, the gold-coated silver nanoplates contained in the gold-coated silver nanoplate suspension A2 were a mixture of plates having circular shapes and polygonal shapes including triangular shapes in which an average maximum length (particle diameter) of the main surfaces was 18 nm, and had an average thickness of 8 nm. Note that the average maximum length (particle diameter) of the main surfaces of the gold-coated silver nanoplates was determined by measuring maximum lengths (particle diameters) of any 100 gold-coated silver nanoplates in a SEM image (captured using a scanning electron microscope SU-70 manufactured by Hitachi, Ltd.) to obtain a total of 100 pieces of data, and excluding the highest and lowest 10% of the data to thus take the average of 80 sites for use as the maximum length (particle diameter) (the same shall also apply to gold-coated silver nanoplate suspensions B2 and C to be described later). Moreover, an average thickness of gold on the gold-coated silver nanoplates contained in the gold-coated silver nanoplate suspension A2 was 0.25 nm according to high-angle annular dark field scanning transmission electron microscopy (HAADF-STEM). Note that the average thickness of gold was determined by selecting any ten gold-coated silver nanoplate particles from a HAADF-STEM image, measuring any ten sites on each of the particles to obtain data on thicknesses of gold at 100 sites in total, and excluding the highest and lowest 10% of the data to thus take the average of 80 sites for use as the average thickness of gold (the same shall also apply to the gold-coated silver nanoplate suspensions B2 and C to be described later).

Further, the gold-coated silver nanoplate suspension A2 had a gold concentration of 0.056 mg/L and a silver concentration of 0.226 mg/L. The gold thickness calculated according to the following calculation method was 0.21 nm.

1. ICP emission spectroscopy result

$$Gold concentration \text{:} silver concentration = 0.056 ( mg \text{/} L ) \text{:} 0.226 ( mg \text{/} L ) = 1 \text{:} 4.04$$

2. Volume of silver nanoplate (shape: equilateral triangle, particle diameter: 18 nm, thickness: 8 nm)

$$( area of triangle ) \times ( thickness ) = ( 18 nm \times ( 18 √ 3 / 2 ) nm \div 2 ) \times 8 nm = 1122 {nm}^{3} ( = 1122 \times {10}^{- 21} {cm}^{3} )$$

3. Relative Density of Silver

10.51 q/cm³

4. Mass of triangular silver nanoplate

$$( volume of equilateral triangular silver nanoplate ) \times ( relative density of silver ) = ( 1122 \times {10}^{- 21} {cm}^{3} ) \times 10.51 g \text{/} {cm}^{3} = 1.18 \times {10}^{- 17} g$$

5. Mass (X) of gold coating triangular silver nanoplate

1:4.04=X:1.18×10⁻¹⁷ g

X=0.29×10⁻¹⁷ g

6. Relative Density of Gold

19.32 g/cm³

7. Volume of gold coating triangular silver nanoplate

$$( mass of gold ) \div ( relative density of gold ) = 0.29 \times {10}^{- 17} g \div 19.32 g \text{/} {cm}^{3} = 1.51 \times {10}^{- 19} {cm}^{3} ( = 151 {nm}^{3} )$$

8. Surface area of triangular silver nanoplate

$$( areas of triangular surface ) + ( areas of side surfaces of particle ) = ( 18 nm \times ( 18 √ 3 \text{/} 2 ) nm \div 2 ) \times 2 ) + ( 8 nm \times 18 nm \times 3 ) = 713 {nm}^{2}$$

9. Thickness of gold coating triangular silver nanoplate

$$( volume of gold coating equilateral triangular silver nanoplate ) \div ( surface area of equilateral triangular silver nanoplate ) = 151 {nm}^{3} \div 713 {nm}^{2} = 0.21 nm$$

Note that the gold concentration and the silver concentration were analyzed according to the following procedure (the same shall also apply to the gold-coated silver nanoplate suspensions B2 and C to be described later).

1. After 0.5 mL of the gold-coated silver nanoplate suspension A2 was centrifuged (25,000 rpm, 26,000 g), the supernatant was removed. The resulting precipitate was suspended again in ultrapure water in the same amount as that of the removed supernatant.

2. After 15 mL of aqua regia was added to the suspension obtained in step 1 above, the resultant was boiled for 5 minutes. Thereby, gold and silver were dissolved into aqua regia.

3. The solution obtained in step 2 above was measured using an ICP emission spectrometer (manufactured by Shimadzu Corporation, ICPS-7510).

The gold-coated silver nanoplate suspension A2 had a polystyrenesulfonic acid concentration of 0.98 nM.

The gold-coated silver nanoplate suspension A2 had a PVP concentration of 8.1 μM.

The gold-coated silver nanoplate suspension A2 had a pH of 4.0 at room temperature (20° C.). For the pH measurement, a twin pH meter B-212 manufactured by HORIBA, Ltd. was used (glass electrode method) (hereinafter the same).

The gold-coated silver nanoplate suspension A2 had a silver content of 0.0016% by mass relative to the total mass of the suspension.

### 1-2-2. Preparation of Gold-Coated Silver Nanoplate Suspension B2

To 120 ml of the silver nanoplate suspension B2, 9.1 ml of an aqueous solution of 0.125 mM PVP (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, a gold-coated silver nanoplate suspension B2 was prepared.

The main dispersion medium of the gold-coated silver nanoplate suspension B2 was water.

As a result of the SEM observation, the gold-coated silver nanoplates contained in the gold-coated silver nanoplate suspension B2 were a mixture of plates having circular shapes and polygonal shapes including triangular shapes in which an average maximum length (particle diameter) of the main surfaces was 31 nm, and had an average thickness of 8 nm. Moreover, an average thickness of gold on the gold-coated silver nanoplates contained in the gold-coated silver nanoplate suspension B2 was 0.30 nm according to HAADF-STEM.

Further, the gold-coated silver nanoplate suspension B2 had a gold concentration of 0.045 mg/L and a silver concentration of 0.185 mg/L. The gold thickness calculated according to the above-described calculation method based on the ICP emission spectroscopy result was 0.28 nm.

The gold-coated silver nanoplate suspension B2 had a polystyrenesulfonic acid concentration of 0.98 nM.

The gold-coated silver nanoplate suspension B2 had a PVP concentration of 8.1 μM.

The gold-coated silver nanoplate suspension B2 had a pH of 4.0 at room temperature (20° C.)

The gold-coated silver nanoplate suspension B2 had a silver content of 0.0016% by mass relative to the total mass of the suspension.

### 1-2-3. Preparation of Gold-Coated Silver Nanoplate Suspension C2

To 120 ml of the silver nanoplate suspension C2, 9.1 ml of an aqueous solution of 0.125 mM PVP (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, a gold-coated silver nanoplate suspension C2 was prepared.

The main dispersion medium of the gold-coated silver nanoplate suspension C2 was water.

As a result of the SEM observation, the gold-coated silver nanoplates contained in the gold-coated silver nanoplate suspension C2 were a mixture of plates having circular shapes and polygonal shapes including triangular shapes in which an average maximum length (particle diameter) of the main surfaces was 50 nm, and had an average thickness of 10 nm. Moreover, an average thickness of gold on the gold-coated silver nanoplates contained in the gold-coated silver nanoplate suspension B2 was 0.45 nm according to HAADF-STEM.

Further, the gold-coated silver nanoplate suspension C2 had a gold concentration of 0.047 mg/L and a silver concentration of 0.186 mg/L. The gold thickness calculated according to the above-described calculation method based on the ICP emission spectroscopy result was 0.41 nm.

The gold-coated silver nanoplate suspension C2 had a polystyrenesulfonic acid concentration of 0.98 nM.

The gold-coated silver nanoplate suspension C2 had a PVP concentration of 8.1 μM.

The gold-coated silver nanoplate suspension C2 had a pH of 4.0 at room temperature (20° C.)

The gold-coated silver nanoplate suspension C2 had a silver content of 0.0016% by mass relative to the total mass of the suspension.

### 1-3. pH Adjustment of Gold-Coated Silver Nanoplate Suspensions

1-3-1. Preparation of pH-Adjusted Suspension of Gold-Coated

### Silver Nanoplates (Suspension D2)

To 1.95 mL of the gold-coated silver nanoplate suspension A2 obtained in 1-2-1, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension D2).

The main dispersion medium of the suspension D2 was water.

The gold-coated silver nanoplates contained in the suspension D2 were a mixture of plates having circular shapes and polygonal shapes including triangular shapes in which an average maximum length of the main surfaces was 18 nm, and had an average thickness of 8 nm. Moreover, an average thickness of gold on the gold-coated silver nanoplates contained in the suspension D2 was 0.25 nm according to HAADF-STEM.

The color tone of a 3-fold diluted solution of the suspension D2 was yellow.

The suspension D2 had a polystyrenesulfonic acid concentration of 0.94 nM.

The suspension D2 had a PVP concentration of 7.8 μM.

The suspension D2 had a pH of 7.3 at room temperature (20° C.).

The suspension D2 had a silver content of 0.0015% by mass relative to the total mass of the suspension.

1-3-2. Preparation of pH-Adjusted Suspension of Gold-Coated Silver Nanoplates (Suspension E2)

To 1.95 mL of the gold-coated silver nanoplate suspension B2 obtained in 1-2-2, 0.05 mL of a 200 mM PBS buffer solution (+ or −) and 0.025 mL of an aqueous solution of 190 mM sodium carbonate were added with stirring. Thus, a pH-adjusted suspension of gold-coated silver nanoplates was prepared (hereinafter, suspension E2). Here, the 200 mM PBS buffer solution (+) refers to a PBS buffer solution containing divalent ions (1.0 mM Mg²⁺ and 1.8 mM Ca²⁻), while the 200 mM PBS buffer solution (−) refers to a PBS buffer solution containing no divalent ion.

The main dispersion medium of the suspension E2 was water.

The gold-coated silver nanoplates contained in the suspension E2 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension E2 was 0.30 nm according to HAADF-STEM.

The color tone of a 3-fold diluted solution of the suspension E2 was magenta.

The suspension E2 had a polystyrenesulfonic acid concentration of 0.94 nM.

The suspension E2 had a PVP concentration of 7.8 μM.

The suspension E2 had a pH of 7.3 at room temperature (20° C.).

The suspension E2 had a silver content of 0.0015% by mass relative to the total mass of the suspension.

1-3-3. pH-Adjusted Suspension of Gold-Coated Silver Nanoplates (Suspension F2)

To 1.95 mL of the gold-coated silver nanoplate suspension C2 obtained in 1-2-3, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension F2).

The main dispersion medium of the suspension F2 was water.

The gold-coated silver nanoplates contained in the suspension F2 were a mixture of plates having circular shapes and polygonal shapes including triangular shapes in which an average maximum length of the main surfaces was 50 nm, and had an average thickness of 10 nm. Moreover, an average thickness of gold on the gold-coated silver nanoplates contained in the suspension F2 was 0.45 nm according to HAADF-STEM.

The color tone of a 3-fold diluted solution of the suspension F2 was cyan.

The suspension F2 had a polystyrenesulfonic acid concentration of 0.94 nM.

The suspension F2 had a PVP concentration of 7.8 μM.

The suspension F2 had a pH of 7.3 at room temperature (20° C.)

The suspension F2 had a silver content of 0.0015% by mass relative to the total mass of the suspension.

2. Supporting of Specific Binding Substance onto Gold-Coated Silver Nanoplates.
2-1. Supporting of Sugar Chain onto Gold-Coated Silver Nanoplates 2-1-1. Supporting of Mercaptoethyl Mannose onto Gold-Coated Silver Nanoplates (Suspension E2). To a 9-mL-volume vial containing 4 mL of the suspension E2 (which was prepared with the PBS buffer solution (+)), 10 mg of 1-mercaptoethyl mannose (Mw: 270.274) was added and stirred at 30° C. for 12 hours at 500 rpm by using a magnetic stirrer. Thus, a suspension G2 of gold-coated silver nanoplates supporting mannose was prepared.

Note that 1-mercaptoethyl mannose was synthesized by organic chemical processes. To be more specific, 1-mercaptoethyl mannose was synthesized through acetylation, bromoethylation, thioacetylation, and deacetylation of position 1 of raw material mannose (manufactured by Kanto Chemical Co., Inc.). The chemical formula of 1-mercaptoethyl mannose is shown below.

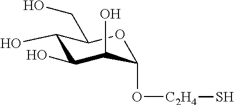

2-2. Supporting of Antibody onto Gold-Coated Silver Nanoplates
2-2-1. Supporting of Anti-Hepatitis B Virus Antigen Antibody onto Gold-Coated Silver Nanoplates

First, 2 mL of a solution of an antibody (product name: Goat anti HBsAg, manufactured by: Arista Biologicals, Inc.) (hereinafter also referred to as anti-HBs antigen antibody) against a hepatitis B virus antigen (HBs antigen) at a concentration of 50 μg/mL in a 5 mM PBS(−) buffer solution was mixed with 18 mL of the suspension D2. The obtained mixture was shaken at room temperature for 30 minutes. Then, the resultant was centrifuged (25000 rpm, 4° C., 10 minutes) to precipitate the gold-coated silver nanoplates supporting the antibody, and 18.5 mL of the supernatant was removed. Subsequently, the gold-coated silver nanoplates supporting the antibody were dispersed again in 18.5 mL of a 5 mM PBS(−) buffer solution. Thus, a suspension H2 of gold-coated silver nanoplates supporting the anti-HBs antigen antibody was prepared.

The suspension E2 (which was prepared with the PBS buffer solution (−)) and the suspension F2 were also caused to support the antibody in the same manner. Thus, suspensions I2 and J2 of gold-coated silver nanoplates supporting the anti-HBs antigen antibody were prepared.

The gold-coated silver nanoplates supporting the anti-HBs antigen antibody in the suspensions H2, I2, and J2 had maximum absorption wavelengths of 458 nm, 532 nm, and 630 nm, respectively.

The prepared suspensions H2, I2, and J2 were stably dispersed in the buffer solutions, and also the spectral properties hardly changed. This revealed that all of these are highly stable against oxidation.

3. Interaction with Test Substances

### 3-1. Interaction Between Lectin and Gold-Coated Silver Nanoplates Supporting Sugar Chain

Into each of five Eppendorf tubes, 500 μL of the suspension

G2 (PBS(+)) of gold-coated silver nanoplates supporting mannose was dispensed. Then, solutions of concanavalin A (abbreviated as: ConA, product name: concanavalin A, manufactured by: J-OIL MILLS, Inc.) at a concentration of 10 μM in a 5 mM PBS (+) buffer solution were added in amounts of 5 μL (amount of substance of ConA: 50 pmol, the concentration after the addition: 0.1 μM), 10 μL (amount of substance of ConA: 100 pmol, the concentration after the addition: 0.2 μM), 25 μL (amount of substance of ConA: 250 pmol, the concentration after the addition: 0.5 μM), and 50 μL (amount of substance of ConA: 500 pmol, the concentration after the addition: 1.0 μM) to the Eppendorf tubes, respectively, followed by stirring, and then left standing at 30° C. for 1 hour.

### (1) Color-Tone Change and Precipitate Formation

While the suspension G2 of gold-coated silver nanoplates supporting mannose was left standing after ConA (50 μL) was added thereto, the color-tone change of the suspension and the precipitate formation in the suspension were visually observed. Table 4 shows the result.

TABLE 4
Color-tone change of suspension and precipitate formation

standing time

| | | 7 minutes | 17 minutes |
|---|---|---|---|
| | 0 minutes | later | later |
| color | magenta | light pink | gray |
| precipitate | absent | absent | present |

### (2) Extinction Measurement

The extinction of each of the interaction solutions was measured using a UV-Vis-NIR spectrometer MPC3100UV-3100PC manufactured by Shimadzu Corporation under conditions of an optical path length: 1 cm and a measurement wavelength: 300 to 1000 nm. FIG. 24 and Table 5 show the measurement result.

TABLE 5
Amount of ConA added and extinction change

| | Maximum | | Percent of |
|---|---|---|---|
| Amount of ConA | absorption | | extinction |
| added | wavelength | Extinction | decreased |
| (pmol) | (nm) | (Abs.) | (%) |
| 0 | 526 | 2.78 | 0.0% |
| 50 | 524 | 2.61 | 6.1% |
| 100 | 524 | 2.50 | 10.1% |
| 250 | 522 | 1.79 | 35.5% |
| 500 | 534 | 0.65 | 76.7% |

Even when ConA was added to the suspension of gold-coated silver nanoplates supporting no sugar chain, the extinction did not change (the data is not shown). In contrast, when ConA was added to the suspension G2 of gold-coated silver nanoplates supporting mannose, the extinction was decreased depending on the amount of ConA added. This indicates a change because of the formation of the complex of ConA with the gold-coated silver nanoplates supporting mannose.

### 3-2. Interaction Between Antigen and Gold-Coated Silver Nanoplates Supporting Antibody

Into each of two 9-mL-volume vials, 5 mL of the suspension H2 (PBS (−)): gold-coated silver nanoplates supporting the anti-HBs antibody was dispensed. Then, 50 μL of a solution of a hepatitis B virus antigen (abbreviated as: HBsAg, product name: HBsAg Protein (Subtype adr), manufactured by: Fitzgerald Industries International Inc.) at a concentration of 5 μM in a 5 mM PBS(−) buffer solution (amount of substance of HBs antigen: 250 pmol, the concentration after the addition: 50 nM), and 50 μL of a solution of a hepatitis B virus antigen at a concentration of 10 μM in a 5 mM PBS (−) buffer solution (amount of substance of HBs antigen: 500 pmol, the concentration after the addition: 100 nM) were added to the vials, respectively, followed by stirring, and then left standing at 30° C. for 1 hour.

### (1) Extinction Measurement

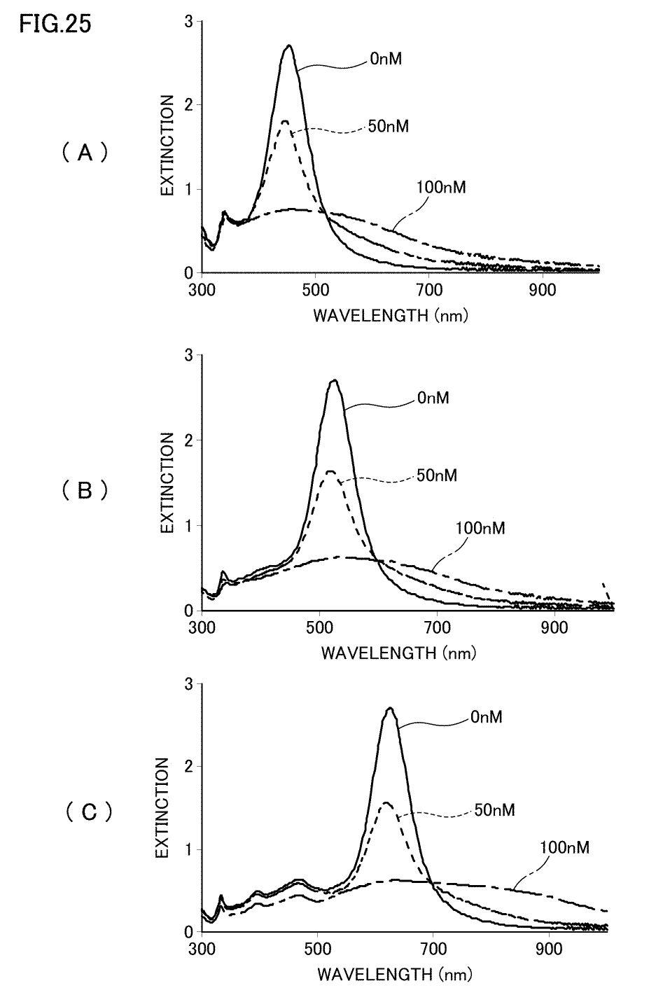

The extinction of each of the interaction solutions prepared from the suspension H2 was measured using a UV-Vis-NIR spectrometer MPC3100UV-3100PC manufactured by Shimadzu Corporation under conditions of an optical path length: 1 cm and a measurement wavelength: 300 to 1000 nm. FIG. 25A shows the measurement result.

In addition, FIGS. 25B and 25C show the result when the suspension I2 or J2 was used.

Even when the HBs antigen was added to the suspension of gold-coated silver nanoplates supporting no antibody, the extinction did not change (the data is not shown). In contrast, when the HBs antigen was added to the suspension H2, I2, or J2 of gold-coated silver nanoplates supporting the anti-HBs antigen antibody, the extinction was decreased depending on the amount of the HBs antigen added. This indicates a change because of the formation of the complex of the HBs antigen with the gold-coated silver nanoplates supporting the anti-HBs antigen antibody.

### (2) Turbidity Measurement

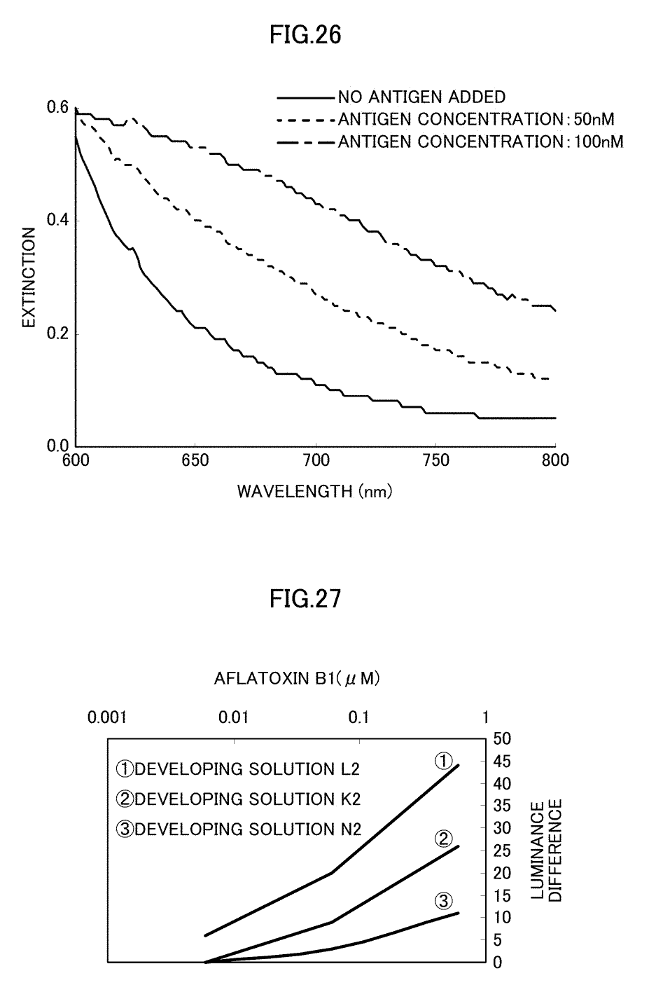

When the HBs antigen was added to the suspension I2 of gold-coated silver nanoplates supporting the anti-HBs antigen antibody, the extinctions at wavelengths around 700 nm were measured as the turbidities of the suspension. FIG. 26 shows the measurement result, which is a partially enlarged graph of FIG. 25B.

Even when the HBs antigen was added to the suspension of gold-coated silver nanoplates supporting no antibody, the turbidity did not change (the data is not shown). In contrast, when the HBs antigen was added to the suspension I2 of gold-coated silver nanoplates supporting the anti-HBs antigen antibody, the turbidity was increased depending on the amount of the HBs antigen added. This indicates a change because of the formation of the complex of the HBs antigen with the gold-coated silver nanoplates supporting the anti-HBs antigen antibody.

### Example 12

### 1. Preparation of Gold-Coated Silver Nanoplates

### 1-1. Preparation of Gold-Coated Silver Nanoplate Suspension (Suspension K2)

To 120 ml of the silver nanoplate suspension B2 prepared in 1-1-3 of Example 11, 9.1 ml of an aqueous solution of 0.125 mM PVP (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, a suspension K2 of gold-coated silver nanoplates was prepared.

The main dispersion medium of the suspension K2 was water.

The gold-coated silver nanoplates contained in the suspension K2 were a mixture of plates having circular shapes and polygonal shapes including triangular shapes in which an average maximum length (particle diameter) of the main surfaces was 31 nm, and had an average thickness of 8 nm. Moreover, an average thickness of gold on the gold-coated silver nanoplates contained in the suspension K2 was 0.30 nm.

The suspension K2 bad a polystyrenesulfonic acid (corresponding to the water-soluble polymer in the present invention) concentration of 0.98 nM.

The suspension K2 had a PVP concentration of 8.1 μM. Note that the average thickness of gold was determined by selecting any ten gold-coated silver nanoplate particles from a HAADF-STEM image, measuring any ten sites on each of the particles to obtain data on thicknesses of gold at 100 sites in total, and excluding the highest and lowest 10% of the data to thus take the average of 80 sites for use as the average thickness of gold.

The suspension K2 had a pH of 4.0 at room temperature (20° C.). For the pH measurement, a twin pH meter B-212 manufactured by HORIBA, Ltd. was used (glass electrode method) (hereinafter the same).

The suspension K2 had a silver content of 0.0016% by mass relative to the total mass of the suspension.

1-2. Preparation of pH-Adjusted Suspension of Gold-Coated Silver Nanoplates (Suspension L2)

To 1.95 mL of the suspension K2 prepared in 1-1 above, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension L2).

The main dispersion medium of the suspension L2 was water.

The gold-coated silver nanoplates contained in the suspension L2 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension L2 was 0.30 nm.

The suspension L2 had a polystyrenesulfonic acid concentration of 0.94 nM.

The suspension L2 had a PVP concentration of 7.8 μm.

The suspension L2 had a pH of 7.3 at room temperature (20° C.).

The suspension L2 had a silver content of 0.0015% by mass relative to the total mass of the suspension.

1-3. Preparation of pH-Adjusted Suspension of Gold-Coated Silver Nanoplates (Suspension M2)

To 120 ml of the silver nanoplate suspension B2 prepared in 1-1-3 of Example 11, 9.1 ml of an aqueous solution of 1.25 mM PVP (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and 0.025 mL of an aqueous solution of 190 mM sodium carbonate were added with stirring. Thus, a pH-adjusted suspension of gold-coated silver nanoplates was prepared (hereinafter, suspension M2).

The main dispersion medium of the suspension M2 was water.

The gold-coated silver nanoplates contained in the suspension M2 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension M2 was 0.30 nm.

The suspension M2 had a polystyrenesulfonic acid concentration of 0.94 nM.

The suspension M2 had a PVP concentration of 78 μM.

The suspension M2 had a pH of 7.3 at room temperature (20° C.)

The suspension M2 had a silver content of 0.0015% by mass relative to the total mass of the suspension.

1-4. Preparation of pH-Adjusted Suspension of Gold-Coated Silver Nanoplates (Suspension N2)

To 120 ml of the silver nanoplate suspension B2 prepared in 1-1-3 of Example 11, 9.1 ml of an aqueous solution of 0.125 mM PVP (molecular weight: 40,000) was added, and 1.6 ml of an aqueous solution of 0.5 M ascorbic acid was added. Then, 9.6 ml of an aqueous solution of 0.42 mM chloroauric acid was added thereto with stirring at 0.5 mL/min. The obtained solution was left standing in an incubator (30° C.) for 24 hours. Thereby, an aqueous suspension of gold-coated silver nanoplates was prepared in which the surfaces of the silver nanoplates were coated with gold.

To 1.95 mL of the prepared aqueous suspension of gold-coated silver nanoplates, 0.05 mL of a 200 mM PBS buffer solution and an aqueous solution of 200 mM sodium hydroxide were added with stirring. Thus, a pH-adjusted suspension of gold-coated silver nanoplates was prepared (hereinafter, suspension N2).

The main dispersion medium of the suspension N2 was water.

The gold-coated silver nanoplates contained in the suspension N2 were a mixture of plates having circular shapes and polygonal shapes including triangular shapes in which an average maximum length of the main surfaces was 31 nm, and had an average thickness of 8 nm. Moreover, an average thickness of gold on the gold-coated silver nanoplates contained in the suspension N2 was 0.30 nm.

The suspension N2 had a polystyrenesulfonic acid concentration of 0.94 nM.

The suspension N2 had a PVP concentration of 7.8 μM.

The suspension N2 had a pH of 11.5 at room temperature (20° C.)

The suspension N2 had a silver content of 0.0015% by mass relative to the total mass of the suspension.

2. Supporting of Antibody onto Gold-Coated Silver Nanoplates and Preparation of Developing Solutions Used in Immunochromatography

First, 0.2 mL of a solution of an antibody (product name: AFB1 (Aflatoxin B1) Antibody, manufactured by: IMMUNE CHEM) against aflatoxin B1 at a concentration of 50 μg/mL in a 5 mM PBS (−) buffer solution was mixed with 1.8 mL of the suspension of gold-coated silver nanoplates (the suspension K2, L2, M2, or N2). The obtained mixture was shaken at room temperature for 30 minutes. Then, the resultant was centrifuged (25000 rpm, 4° C., 10 minutes) to precipitate the gold-coated silver nanoplates supporting the antibody, and 1.85 mL of the supernatant was removed. Subsequently, the gold-coated silver nanoplates supporting the antibody were dispersed again in a 5 mM PBS(−) buffer solution containing 4.9 μM BSA. The resultant was adjusted to have an extinction of 2.0 by using spectrometer (apparatus name: UV-Vis-NIR spectrometer MPC3100UV-3100PC, manufactured by: Shimadzu Corporation). In this manner, suspensions of developing solutions K2, L2, M2, and N2 were prepared. The developing solutions K2, L2, M2, and N2 had maximum absorption wavelengths of 532 nm. Table 6 shows relations between the prepared developing solutions and the suspensions of gold-coated silver nanoplates.

TABLE 6
Relations between prepared developing solutions and
suspensions of gold-coated sliver nanoplates

| | Suspension of gold-coated |
|---|---|
| Developing solution | silver nanoplates |
| Developing solution K2 | Suspension K2 |
| Developing solution L2 | Suspension L2 |
| Developing solution M2 | Suspension M2 |
| Developing solution N2 | Suspension N2 |

3. Detection of Interaction with Test Substance by Immunochromatography (Part 1)

According to the same procedure as in (2) of Example 1, an immunochromatography with the test substance of aflatoxin B1 was conducted using an immunochromatographic test paper having an anti-aflatoxin B1 antibody (capture antibody) immobilized in a straight line (the detection line of FIG. 10).

The immunochromatographic test paper used was purchased from a contractor company (BioDevice Technology, Co., Ltd.) for preparing immunochromatographic test papers. When the capture antibody was immobilized in a straight line, a solution was used in which the anti-aflatoxin B1 antibody was adjusted to a concentration of 1 g/mL by using a 5 mM PBS (−) buffer solution.

A first developing solution (developing solution used in FIG. 10(a)) was a solution of aflatoxin E1 (product name: aflatoxin B1, manufactured by: Toronto Research Chemicals Inc.) in a 5 mM PBS(−) buffer solution. As the first developing solution, solutions were prepared which had aflatoxin B1 concentrations of 0.60 μM, 0.06 μM, 0.006 μM, and 0 M (blank), respectively.

A second developing solution was a 5 mM PBS(−) buffer solution.

A third developing solution (developing solution used in FIG. 10(c)) was the developing solution K2, L2, M2, or N2 prepared in 2 above.

The specific test procedure was as follows.

On the immunochromatographic test paper, 15 μL of the first developing solution was developed (FIG. 10(a)). As the first developing solution was developed, aflatoxin B1 was captured by the capture antibody immobilized on the detection line of the test paper (FIG. 10(b)).

Then, 30 μL of the second developing solution was developed to wash away excessive antigens on the immunochromatographic test paper.

Finally, 60 μL of the third developing solution was developed (FIG. 10(c)). As the third developing solution was developed, the detection antibody (the gold-coated silver nanoplates supporting the anti-aflatoxin B1 antibody) bound to aflatoxin B1 captured on the detection line of the test paper (FIG. 10(d)).

The same procedure was repeated using each of the first developing solutions having different aflatoxin B1 concentrations.

### (1) Immunochromatography Evaluation by Visual Judgment

After the third developing solution was developed, the coloring (magenta) of the gold-coated silver nanoplates on the detection line was visually checked to judge the presence or absence of aflatoxin B1. Table 7 shows the result.

TABLE 7
Visual judgment result of immunochromatography

| Aflatoxin B1 | | | | |
|---|---|---|---|---|
| concentration | | | | |
| (μM) in the | | | | |
| first | Developing | Developing | Developing | Developing |
| developing | solution | solution | solution | solution |
| solution | K2 | L2 | M2 | N2 |
| 0.6 | + | + | + | + |
| 0.06 | + | + | + | + |
| 0.006 | − | + | − | − |
| 0 (blank) | − | − | − | − |

+: the coloring on the detection line was observed.
−: the coloring on the detection line was not observed.

Even when the suspension of gold-coated silver nanoplates supporting no antibody was used as the third developing solution, the color of the detection line did not change (the data is not shown). In contrast, when the suspension of gold-coated silver nanoplates supporting the anti-aflatoxin B1 antibody was used as the third developing solution, the detection line was colored in magenta. This is because the gold-coated silver nanoplates supporting the anti-aflatoxin B1 antibody formed a complex with aflatoxin B1 captured by the capture antibody on the detection line. In addition, only when the developing solution L2 was used, 0.006 μM of aflatoxin B1 was detected.

### (2) Immunochromatography Evaluation by Luminance Analysis

After the third developing solution was developed, the immunochromatographic test paper was scanned (apparatus name: Cano Scan LiDE500F, manufactured by: Canon Inc.) to quantify the lowest luminance at the detection line (immobilized section of the capture antibody) and the lowest luminance at a section other than the detection line by using image analysis software (Image-J: open-source, public-domain image processing software (http://imagej.nih.gov/ij/) developed by Wayne Rasband at the National Institutes of Health). The lowest luminance was determined by measuring five times luminances at different positions in the target region (the detection line or the section other than the detection line) and adopting a median of the obtained numerical values as the lowest luminance. The luminance difference was calculated according to the following equation:

Luminance difference=the lowest luminance at the detection line−the lowest luminance at the section other than the detection line.

### (2-1) Influence of pH

Table 8 and FIG. 27 show the luminance differences when the developing solution K2, L2, or N2 was used.

TABLE 8
Luminance analysis result of immunochromatography

| Aflatoxin B1 | Developing | Developing | Developing |
|---|---|---|---|
| concentration (μM) in the | solution | solution | solution |
| first developing solution | K2 | L2 | N2 |
| 0.6 | 96 | 44 | 11 |
| 0.06 | 9 | 20 | 3 |
| 0.006 | 0 | 6 | 0 |
| 0 (blank) | 0 | 0 | 0 |

Numerical values in the column of each developing solution indicate luminance differences.

Even when the suspension of gold-coated silver nanoplates supporting no antibody was used as the third developing solution, no luminance difference was found (the data is not shown). In contrast, when the suspension of gold-coated silver nanoplates supporting the anti-aflatoxin B1 antibody was used as the third developing solution, a remarkable luminance difference was measured. This is because the gold-coated silver nanoplates supporting the anti-aflatoxin B1 antibody formed a complex with aflatoxin B1 captured by the capture antibody on the detection line. Moreover, the developing solution L2 prepared from the suspension L2 having a pH of 7.3 or the developing solution K2 prepared from the suspension K2 having a pH of 4.0 had a higher luminance difference than that of the developing solution N2 prepared from the suspension N2 having a pH of 11.5. This revealed that the former has higher detection sensitivity than the latter. When the developing solution L2 was used, a luminance difference was found even with 0.006 μM of aflatoxin B1. This revealed that the developing solution L2 has a particularly high detection sensitivity.

### (2-2) Influence of Water-Soluble Polymer

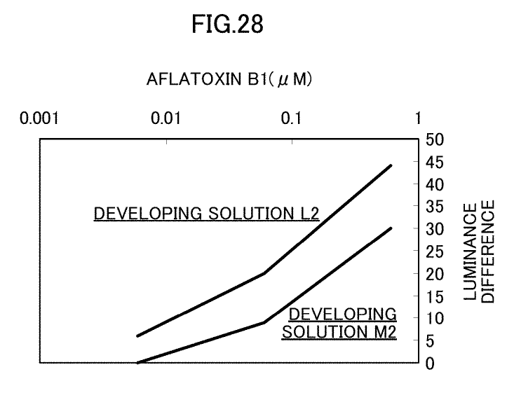

Table 9 and FIG. 28 show the luminance differences when the developing solution L2 or M2 was used.

TABLE 9
Luminance analysis result of immunochromatography

| Aflatoxin B1 | | |
|---|---|---|
| concentration (μM) in | | |
| the first developing | Developing | Developing |
| solution | solution L2 | solution M2 |
| 0.6 | 44 | 30 |
| 0.06 | 20 | 9 |
| 0.006 | 6 | 0 |
| 0 (blank) | 0 | 0 |

Numerical values in the column of each developing solution indicate luminance differences.

The developing solution L2 prepared from the suspension L2 having a water-soluble polymer PVP concentration of 7.8 μM had a luminance difference even with 0.006 μM of aflatoxin B1. This revealed that the developing solution L2 has a higher detection sensitivity than that of the developing solution M2 prepared from the suspension M2 having a PVP concentration of 78 μM.

From the foregoing, it was found out that the suspension of gold-coated silver nanoplates supporting a specific binding substance for a test substance of the present invention is prepared as a stable suspension, applicable for detecting various test substances, compatible with various detection mean, and capable of detecting the test substances with high sensitivity.

### INDUSTRIAL APPLICABILITY

The present invention is utilizable in the field of the detection technique in which gold-coated silver nanoplates are used as labels. The use of the suspension of the present invention enables accurate determinations in detecting a wide variety of test substances.

1.-14. (canceled)

15. A suspension of gold-coated silver nanoplates, the suspension comprising 0 to 50 μM of a water-soluble polymer and having a pH of 10 or less.

16. The suspension according to claim 15, wherein an average thickness of gold on the gold-coated silver nanoplates is 1.0 nm or less.

17. The suspension according to claim 15, wherein an average thickness of gold on the gold-coated silver nanoplates is 0.1 to 0.7 nm.

18. The suspension according to claim 15, wherein the concentration of the water-soluble polymer is 0 to 25 μM.

19. The suspension according to claim 15, wherein the pH is 4 to 10.

20. The suspension according to claim 15, wherein the pH is 5 to 9.

21. The suspension according to claim 15, wherein the gold-coated silver nanoplates support a specific binding substance for a test substance.

22. The suspension according to claim 21, wherein a combination of the test substance and the specific binding substance, respectively, is selected from the group consisting of an antigen and an antibody capable of binding thereto, an antibody and an antigen capable of binding thereto, a sugar chain or a glycoconjugate and a lectin capable of binding to the sugar chain or the glycoconjugate, a lectin and a sugar chain or a glycoconjugate capable of binding to the lectin, a hormone or a cytokine and a receptor capable of binding to the hormone or the cytokine, a receptor and a hormone or a cytokine capable of binding to the receptor, a protein and a nucleic acid aptamer or a peptide aptamer capable of binding to the protein, an enzyme and a substrate capable of binding thereto, a substrate and an enzyme capable of binding thereto, biotin and avidin or streptavidin, avidin or streptavidin and biotin, IgG and Protein A or Protein G, Protein A or Protein G and IgG, and a first nucleic acid and a second nucleic acid capable of binding thereto.

23. A method for detecting the test substance by using the suspension according to claim 21, the method comprising the steps of:
mixing the suspension with the test substance to form a complex of the test substance with the gold-coated silver nanoplates supporting the specific binding substance; and
detecting the complex.

24. The method according to claim 23, wherein formation of the complex is detected by means selected from the group consisting of extinction measurement, absorbance measurement, turbidity measurement, particle size distribution measurement, particle diameter measurement, Raman scattering measurement, color-tone change observation, aggregate- or precipitate-formation observation, immunochromatography, electrophoresis, and flow cytometry.

25. The method according to claim 23, wherein formation of the complex is detected by extinction measurement or absorbance measurement at an absorption wavelength of the gold-coated silver nanoplates within a range of 200 to 2500 nm.

26. The method according to claim 23, wherein formation of the complex is detected by extinction measurement or absorbance measurement at a maximum absorption wavelength of the gold-coated silver nanoplates within a range of 430 to 2000 nm.

27. The method according to claim 23, wherein formation of the complex is detected by turbidity measurement in a wavelength region which is a long-wavelength side of a maximum absorption wavelength of the gold-coated silver nanoplates, and in which an extinction or absorbance is increased depending on the formation of the complex.

28. A kit for use in the method according to claim 23, the kit comprising the suspension according to claim 21.

29. A suspension of gold-coated silver nanoplates, the suspension comprising more than 0 to 50 μM of a water-soluble polymer and having a pH of 10 or less.

30. The suspension according to claim 29, wherein the gold-coated silver nanoplates support a specific binding substance for a test substance.

31. A method for detecting the test substance by using the suspension according to claim 30, the method comprising the steps of:
mixing the suspension with the test substance to form a complex of the test substance with the gold-coated silver nanoplates supporting the specific binding substance; and
detecting the complex.

32. A kit for use in the method according to claim 31, the kit comprising the suspension according to claim 30.
